(19) United States
(12) Patent Application Publication
(10) Pub. No.: US 2024/0197577 A1
(43) Pub. Date: Jun. 20, 2024

(54) SELF-ADHESIVE DENTAL MATERIAL

(71) Applicant: Kulzer GmbH, Hanau (DE)

(72) Inventors: Maria LECHMANN-DORN, Frankfurt (DE); Michael GERLACH, Hofheim (DE)

(73) Assignee: Kulzer GmbH, Hanau (DE)

(21) Appl. No.: 18/285,275

(22) PCT Filed: Mar. 31, 2022
(86) PCT No.: PCT/EP2022/058709

§ 371 Date: Oct. 2, 2023

(30) Foreign Application Priority Data
Apr. 2, 2021 (DE) 10 2021 108 476.8
Apr. 2, 2021 (DE) 10 2021 108 477.6

(51) Int. Cl. A61K 6/887; A61K 6/54; A61K 6/62; A61K 6/77

(52) U.S. Cl. CPC A61K 6/887; A61K 6/54; A61K 6/62; A61K 6/77

(57) ABSTRACT

A self-adhesive, radiation-curable, dental material comprising A) a monomer component comprising a difunctional urethane (meth)acrylate, hydrophilic alkylene oxide-based di-functional (meth)acrylate-based crosslinkers, at least one acidic monomer of an olefinic acidic monoester of a phosphoric acid and/or an olefinic acidic monoester of a thiophosphoric acid optionally in combination with an olefinic carboxylic acid and/or olefinic carboxylic acid anhydrides and an initiator and/or an initiator system having an absorption maximum of from 420 to 550 nm and optionally having a co-initiator and optionally B) a filler component comprising at least one dental glass, wherein in particular A) the monomer component and B) the filler component are present in a mass ratio of a) 100:0 to 60:40 in fissure sealants or b) from 59:41 to 20:80 in composite materials, and wherein the total composition of the fissure sealant or composite material comprises 100 wt.- %.

A self-adhesive, radiation-curable dental material comprising A) a monomer component comprising a difunctional urethane (meth)acrylate, hydrophilic alkylene oxide-based di-functional (meth)acrylate-based crosslinkers, at least one acidic monomer of an olefinic acidic monoester of a phosphoric acid and/or an olefinic acidic monoester of a thiophosphoric acid optionally in combination with an olefinic carboxylic acid and/or olefinic carboxylic acid anhydride and an initiator and/or an initiator system having an absorption maximum of 420 to 550 nm and optionally with a co-initiator and optionally B) a filler component comprising at least one dental glass, wherein in particular A) the monomer component and B) the filler component are present in a mass ratio of a) 100:0 to 60:40 in fissure sealants or b) from 59:41 to 20:80 in composite materials, and wherein the total composition of the fissure sealant or composite material comprises 100 wt.- %.

Many dental composites are known that are universally applicable for direct adhesive restorations as well as for the extraoral fabrication of indirect dentures. A high filler content is advantageous in order to achieve very good mechanical properties of the cured composite and at the same time to reduce the polymerization shrinkage that occurs during curing. These properties are also decisive for the long-term success of the dental restoration material.

Fissure sealants are preventive products to minimize the risk of caries in deep fissures or in susceptible patients. Fissure sealants are polymerizable compositions that adhere to the enamel. The standard procedure for fissure sealing involves acid etching of the enamel followed by sealing with a low-viscosity material, usually referred to as flow, or a glass ionomer cement—without the prior use of a bonding agent. Alternatively, suitable flow materials can be used in combination with an adhesive.

Object of the invention was to provide a dental, self-adhesive polymerizable dental material which, depending on the filler content, is preferably adjustable as a self-adhesive fissure sealant which bonds adhesively to enamel with high adhesive strength, or as a self-adhesive composite. The dental material to be developed should preferably be applicable without prior chemical treatment of the dentin and/or the enamel, preferably without prior etching of the dentin and/or the enamel or the fissure surface. Furthermore, it should be possible to apply the dental material without prior application of an adhesive to the dentin and/or enamel. Furthermore, it was a further object to develop a self-adhesive dental material with reduced water absorption (μg/mm³) and/or reduced solubility (μg/mm³). In addition, it was an object to develop a Bis-GMA free dental material. Expediently, the dental material is also free of HEMA.

The problems are solved with a self-adhesive dental material according to claim 1 and a polymerized dental material according to claim 21 as well as the composite for use for self-adhesive and radiation-curing adhesion and sealing of dentinal tubules as well as for self-adhesive adhesion to enamel. The subclaims and description provide further detailed disclosures of the articles of the invention.

A self-adhesive composite offers the customer the advantage of using the restorative material on the tooth surface comprising enamel and dentin without prior use of an adhesive system and etchant. The composite according to the invention exhibits high bond strength due to the special tuning of the formulation of the composite, which produces very high bond strength on both enamel and dentin in self-etching mode. The achieved bond strength values are significantly higher than the values achieved by the competitor in the market. Shear bond strengths on bovine tooth measured according to ISO 29022 (TC—artificial aging 5 000 cycles thermal cycling between 5° C. and 55° C. A dental material according to the invention shows a shear bond strength of greater than 15 MPa on dentin and greater than 20 MPa on enamel, while the following products show shear bond strengths of 4.8 to 16.4 MPa under otherwise identical conditions (Vertise Flow (5.9 MPa SBS dentin (SBS, shear bond strength), 16.4±4.2 MPa enamel, with TWL 2.1±2.9 MPa), Constic (4.8 MPa SBS dentin, 15 MPa enamel, with TWL 12.0±5.6 MPa)).

A subject of the invention is a self-adhesive, radiation-curable, polymerizable dental material comprising.
A) a monomer component comprising
a) at least one at least difunctional urethane (meth)acrylate,
b) at least one hydrophilic alkylene oxide-based di-functional (meth)acrylate-based crosslinker which i) does not comprise urethane (meth)acrylate and/or ii) does not comprise aromatic residues, or a mixture of at least two of these crosslinkers,
c) at least one olefinic acid ester of a phosphoric acid and/or at least one olefinic acid ester of a thiophosphoric acid,
d) at least one initiator and/or initiator system each having an absorption maximum of 420 to 550 nm and comprising i) diketone and at least one ii) co-initiator selected from a tert-amine and a 1,2-methylenedioxybenzene, wherein i) diketone and
ii) co-initiator are present with a molar ratio of ii):i) of greater than or equal to 1.1, in particular greater than or equal to 1.15, wherein i) the diketone is present at greater than or equal to 0.2 wt.- % (% in weight), in particular greater than or equal to 0.3 wt.- %, preferably greater than or equal to 0.4 wt.- %, in the total composition of the dental material,
   wherein each independently is a (meth)acrylate selected from methacrylate and acrylate, and, wherein each independently is urethane (meth)acrylate selected from urethane methacrylate and urethane acrylate, and optionally

B) a filler component comprising at least one dental glass or mixtures of dental glasses, the total composition of the dental material being 100 wt.- %.

In alternatively preferred dental materials, A) the monomer component and B) the filler component may be present in the dental material in a mass ratio of 100:0 to 30:70 and in equally preferred alternatives a) from 60:40 to 40:60 or b) from 59:41 to 20:80, and wherein the total composition of the dental material is 100 wt.- %. The dental materials according to the invention can be used without to with a low filler content as fissure sealants and with a high filler content, in particular from about 41 wt.- % in the total composition, as composites.

It is further an object of the invention to provide a dental material comprising c) a mixture of acidic monomers comprising i) at least one olefinic acidic ester of a phosphoric acid and/or at least one olefinic acidic ester of a thiophosphoric acid, in combination with ii) at least one olefinic carboxylic acid and/or at least one olefinic carboxylic acid anhydride.

It is also an object of the invention to provide a self-adhesive, radiation-curable dental composite material, in particular a composite material curable or polymerizable with visible light, comprising
A) a monomer component comprising
a) at least one at least difunctional urethane (meth)acrylate,
b) at least one hydrophilic alkylene oxide-based di-functional (meth)acrylate-based crosslinker which i) does not comprise urethane (meth)acrylate and/or ii) does not comprise aromatic residues, or a mixture of at least two of these crosslinkers,
   optionally at least one tri-, tetra-, penta- and/or hexa-functional (meth)acrylate-based crosslinker which is not a urethane (meth)acrylate,
c) a mixture of acidic monomers comprising i) at least one olefinic acidic ester of a phosphoric acid, in particular a monoester of a phosphoric acid, and/or at least one olefinic acidic ester of a thiophosphoric acid, in particular a monoester of a thiophosphoric acid, preferably at least one monoester of a phosphoric acid with a mono-, di-, tri-, or tetra-(meth)acrylate, at least one monoester of a phosphoric acid with a mono-, di-, tri- or tetra-urethane (meth)acrylate and/or monoester of a thiophosphoric acid with a mono-, di-, tri- or tetra-(meth)acrylate, in combination with ii) at least one olefinic carboxylic acid and/or at least one olefinic carboxylic anhydride, in particular at least one carboxylic acid-functionalized and/or carboxylic anhydride-functionalized (meth)acrylate monomer,
d) at least one initiator and/or initiator system each having an absorption maximum of from 420 to 550 nm and comprising at least i) a diketone and at least one ii) co-initiator selected from a tert-amine and a 1,2-methylenedioxybenzene, wherein i) diketone and ii) co-initiator are present at a molar ratio of ii):i) of greater than or equal to 1.1, in particular greater than or equal to 1.15, wherein i) the diketone is present at greater than or equal to 0.22 wt.- %, in particular greater than or equal to 0.3 wt.- %, in particular greater than or equal to 0.5 wt.- %, in the total composition of the dental material, in particular the co-initiator is present at greater than or equal to 1.0 wt.- % in the total composition of the dental material, and
B) a filler component comprising at least one dental glass or mixtures of dental glasses, in particular dental glass of an average particle size d₅₀ of 0.2 to 2.0 μm, wherein optionally at least one pigment, dye, UV stabilizer and/or Vis stabilizer may be present in A) and/or B), and
wherein A) the monomer component and B) the filler component are present in a mass ratio of 100:0 to 20:80, in particular 100:0 to 30:70, in the dental material, in particular from 59:61 to 30:70 in the composite material, and wherein the total composition of the dental material, preferably of the composite material, is 100 wt.- %.

In this context, it is particularly preferred if i) diketone and ii) co-initiator are present with a molar ratio of ii):i) of greater than or equal to 1.15, in particular greater than or equal to 1.2, wherein i) the diketone is present at greater than or equal to 0.50 wt.- %, in particular greater than or equal to 0.55 wt.- %, in particular greater than or equal to 0.6 wt.- %+/−0.25 wt.- %, in the total composition of the dental material.

Under the designation (meth)acrylate, (meth)acrylate-based or urethane (meth)acrylate, both acrylate, acrylate-based or urethane acrylate and preferably methacrylate, methacrylate-based or urethane methacrylate as well as mixtures thereof are disclosed throughout. Thus, by definition, the aforementioned urethane (meth)acrylates also comprise urethane methacrylate and/or urethane acrylate as (meth)acrylate constituent. Accordingly, carboxylic anhydride-functionalized (meth)acrylate monomers also comprise the respective carboxylic anhydride-functionalized methacrylate and/or carboxylic anhydride-functionalized acrylate monomers. And also monoesters of phosphoric acids based on (meth)acrylates, such as (meth)acryloyloxy derivatives; may be selected from the respective methacrylates and acrylates of the respective compound. This applies throughout to all (meth)acrylates in that they are selected from methacrylates and acrylates.

Even though the dental material is described as a dental material comprising a monomer component A) and a filler component B), these two components are present in the composite as a mixture. This type of disclosure is known to the person skilled in the art in the dental field, since it serves only to characterize the two initially prepared components A) and B) before they are mixed to produce the dental material. The mixing of the two components A) and B) is usually carried out beforehand in a disperser for producing the dental material, such as a fissure sealant or composite.

Preferably, the initiator system comprises an initiator such as a diketone and a co-initiator. Particularly preferred is at least one initiator and/or initiator system having an absorption maximum of 420 to 550 nm each comprising at least a cycloaliphatic diketone and as co-initiator an aromatic tert-amine.

In a particularly preferred embodiment, the dental material comprises in the A) monomer component d) 0.3 to 2.5 wt.- %, in particular from 0.5 to 1.2 wt.- %, of at least one diketone, in particular 1,7,7-trimethyl-bicyclo-[2.2.1]-hepta-2,3-dione (champherquinone), and 0.6 to 2.5 wt.- %, in particular from 1.0 to 2.0 wt.- %, preferably from 1.0 to 1.5 wt.- %, of at least one tert-amine, in particular 2-n-butoxyethyl-4-(dimethylamino)benzoate with respect to the total composition of 100 wt.- % of the A) monomer component. In this case, the mass ratio of diketone to co-initiator is from 1:1 to 1:2.5, in particular from 1:1.5 to 1:2.5, preferably from 1:1.8 to 1:2.5.

According to a preferred embodiment, the dental material d) comprises at least one initiator and/or initiator system comprising i) diketone and ii) co-initiator selected from a tert-amine, present at a molar ratio of ii):i) of greater than or equal to 1.15, in particular greater than or equal to 1.17, preferably greater than or equal to 1.2, and wherein i) the diketone is present at greater than or equal to 0.4 wt.- %, in particular greater than or equal to 0.5 wt.- %, in the total composition of the dental material of 100 wt.- %. In particularly preferred embodiments, the diketone is present from 0.5 to 1.0 wt.- % and the co-initiator from 1.0 to 1.8 wt.- % in the total composition. In alternatives without filler content, the diketone and co-initiator are present from 0.5 to 1.0 wt.- % and the co-initiator from 1.0 to 1.8 wt.- % in the total composition, with the co-initiator always present in excess to the diktone.

Further, a dental material is preferred which comprises:

i) at least one olefinic acid ester of a phosphoric acid and/or at least one olefinic acid ester of a thiophosphoric acid comprising at least one monoester of a phosphoric acid and a (meth)acrylate, monoester of a phosphoric acid and a urethane (meth)acrylate and/or at least one monoester of a thiophosphoric acid and a (meth)acrylate, wherein in each case (meth)acrylate is independently selected from methacrylate and acrylate and independently urethane (meth)acrylate is selected from urethane methacrylate and urethane acrylate. Particularly preferred are the esters of the following formulas I, Ia, Ib and II, with the esters of formulas I and II being further preferred.

Preferred i) olefinic acidic monoesters of a phosphoric acid and/or an olefinic acidic monoester of a thiophosphoric acid comprise acidic monoesters of at least one phosphoric acid comprising at least two urethane (meth)acrylate groups, wherein at least two urethane (meth)acrylate groups are covalently bonded to an O atom of the phosphoric acid by means of a group having at least one trivalent C atom and optionally O atoms and form the monoester of the phosphoric acid, wherein the urethane (meth)acrylate groups are selected from urethane methacrylate and urethane acrylate groups, and wherein each independently is urethane (meth)acrylate selected from urethane methacrylate and urethane acrylate, 2-(meth)acryloyloxyethyl acid phosphate, 2- and 3-(meth)acryloyloxypropyl acid phosphate, 4-(meth)acryloyloxybutylic acid phosphate, 6-(meth)acryloyloxyhexyl acid phosphate, 8-(meth)acryloyloxyoctyl acid phosphate, 10-(meth)acryloyloxydecylic acid phosphate, 12-(meth)acryloyloxydodecylic acid phosphate, bis(2-(meth)acryloyloxyethyl acid phosphate, bis(2 or 3-(meth)acryloyloxypropyl acid phosphate, 2-(meth)acryloyloxyethylphenylic acid phosphate, 2-(meth)acryloyloxyethyl-p-methoxyphenylic acid phosphate, and the corresponding thiophosphonates of the abovementioned (meth)acrylates, in particular wherein the (meth)acryloyl-functionalized acid monomers are each independently selected from methylacryloyl- and acryloyl-functionalized acid monomers, or mixtures of at least two of the abovementioned monoesters, or a mixture of i) and ii).

Also included is a dental material comprising
A) a monomer component comprising
(a) from 40 to 80 wt.- % of at least one at least difunctional urethane (meth)acrylate,
b) 10 to 35 wt.- % of at least one hydrophilic alkylene oxide-based di-functional (meth)acrylate-based crosslinker which i) does not comprise urethane (meth)acrylate and/or
ii) does not comprise aromatic residues, or a mixture of at least two of these crosslinkers,
c) 5 to 30 wt.- % of a mixture of acidic monomers comprising
i) at least one olefinic acid ester of a phosphoric acid and/or at least one olefinic acid ester of a thiophosphoric acid, in combination with
ii) at least one olefinic carboxylic acid and/or at least one olefinic carboxylic acid anhydride,
d) 0.3 to 5 wt.- % of at least one initiator and/or initiator system, each having an absorption maximum of 420 to 550 nm, the content of i) the at least one diketone being greater than or equal to 0.3 wt.- %, and of ii) at least one co-initiator being greater than or equal to 0.6 wt.-% in the monomer component,
e) 0 to 10 wt.- % of at least one tri-, tetra-, penta- and/or hexa-functional (meth)acrylate-based crosslinker which is not a urethane (meth)acrylate,
f) 0 to 5 wt.- % of at least one pigment, dye, UV stabilizer and/or Vis stabilizer, wherein the total composition of monomer component A) is 100 wt.- %, and optionally
B) a filler component comprising
a) 90 to 100 wt.- % of at least one dental glass or mixtures of dental glasses,
b) 0 to 10 wt.- % of inorganic fluorides, metal oxide, mixed oxides
c) 0 to 5 wt.- % of at least one pigment, dye, UV stabilizer and/or Vis stabilizer, the total composition of filler component B) being 100 wt.- %, wherein A) the monomer component and B) the filler component are present in the dental material in a mass ratio of 100:0 to 60:40, and wherein the total composition of the dental material is 100 wt.- %.

A further object of the invention is a dental material comprising
A) a monomer component comprising
(a) from 40 to 80 wt.- % of at least one at least difunctional urethane (meth)acrylate,
b) 10 to 35 wt.- % of at least one hydrophilic alkylene oxide-based di-functional (meth)acrylate-based crosslinker which i) does not comprise urethane (meth)acrylate and/or
ii) does not comprise aromatic residues, or a mixture of at least two of these crosslinkers,
c) 5 to 30 wt.- % of a mixture of acidic monomers comprising
i) at least one olefinic acid ester of a phosphoric acid and/or at least one olefinic acid ester of a thiophosphoric acid, in combination with
ii) at least one olefinic carboxylic acid and/or at least one olefinic carboxylic acid anhydride,
d) 0.3 to 5 wt.- % of at least one initiator and/or initiator system, in each case having an absorption maximum of 420 to 550 nm, the content i) of the at least one diketone being greater than or equal to 0.3 wt.- %, in particular greater than or equal to 0.4 wt.- %, preferably greater than or equal to 0.5 wt.- %, and of the ii) at least one co-initiator being greater than or equal to 0.6 wt.- %, in particular greater than or equal to 0.8 wt.- %, preferably greater than or equal to 1.0 wt.- %, in the monomer component,
e) 0 to 10 wt.- % of at least one tri-, tetra-, penta- and/or hexa-functional (meth)acrylate-based crosslinker which is not a urethane (meth)acrylate,
f) 0 to 5 wt.- % of at least one pigment, dye, UV stabilizer and/or Vis stabilizer, wherein the total composition of monomer component A) is 100 wt.- %, and optionally
B) a filler component comprising
(a) 90 to 100 wt.- % of at least one dental glass or mixtures of dental glasses,
b) 0 to 10 wt.- % of inorganic fluorides, metal oxide, mixed oxides
c) 0 to 5 wt.- % of at least one pigment, dye, UV stabilizer and/or Vis stabilizer,
the total composition of filler component B) being 100 wt.- %,
wherein A) the monomer component and B) the filler component are present in the dental material in a mass ratio of 100:0 to 20:80, and wherein the total composition of the dental material is 100 wt.- %.

According to particularly preferred embodiments, the monoesters comprise monoesters of formulas I, Ia, Ib, II and/or mixtures comprising at least two of these monoesters. According to an alternative embodiment, i) the acidic monoester(s) of at least one phosphoric acid of the general formulas I, Ia, Ib and/or II or mixtures of at least two of the monoesters is/are selected from formulas I, Ia, Ib and II

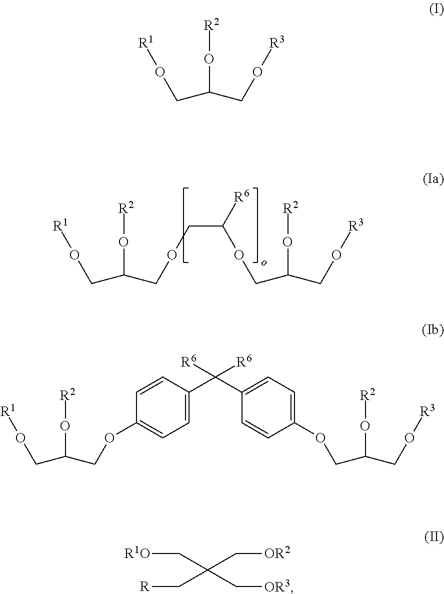

with R=CH₃, OR¹, OR² or OR³
wherein R¹, R² and R³ in formulas I, Ia, Ib and II are each independently selected from formulas III, IIIa, IIIb, IV, V, VI=VIa and/or VIb, VII=VIIa and/or VIIb and VIII=VIIIa and/or VIIIb with

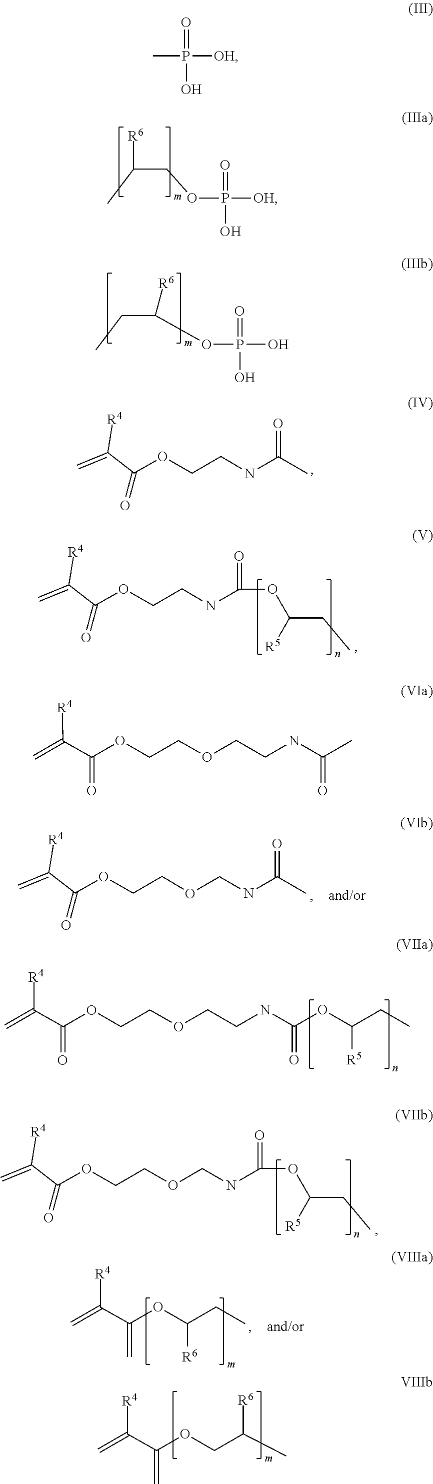

wherein o=1 to 100 in formula Ia, in particular o=1 to 5,
with m=1 to 100 in formulas IIIa and/or IIIb, in particular 1 to 20, wherein in formula IIIa and IIIb R⁶ is selected from H and methyl, and wherein the divalent alkylene oxide group comprising R⁶ may also be present in mirrored or laterally reversed form,
wherein in the formulas V, VIIa and/or VIIb in each case independently n=1 to 100, in particular n=1 to 20, and
in which formulas I, Ia, Ib and II at least one or two residues selected from R¹, R² and R³ correspond to a group of formula III, IIIb and/or IIIa and the remaining residue or residues of R¹, R² and R³ are selected from at least one group of formulas IV, V, VIa, VIb, VIIa, VIIb, VIIIa and/or VIIIb, wherein R⁴ and R⁵ are each independently selected in the groups of formulas IV, V, VIa, VIb, VIIa, VIIb, VIIIa and VIIIb from H, methyl and ethyl, in particular the remaining groups are selected from at least two formulas of IV, V, VIa, VIIb, VIIa, VIIb, VIIIa and/or VIIIb, and
wherein each R⁶ is independently selected from H and methyl in the groups of formulas Ia, Ib, IIIa, IIIb, VIIIa and VIIIb,
wherein m=0 or 1 to 100 in formulas VIIIa and/or VIIIb, in particular 1 to 20, with R⁶ selected from H and methyl.

Typical examples of monophosphoric acid, which may also be present as dual monophosphoric acids, are exemplified below without limiting the disclosure to these examples:

In the monoesters of a phosphoric acid shown below, R^{10A} and R^{10B}/R^{10C} are selected from H and methyl, preferably R^{10A}, R^{10B} and R^{10C} are each H or methyl. In particular with n=0 or 1 to 100, preferably n=0 or 1 to 10.

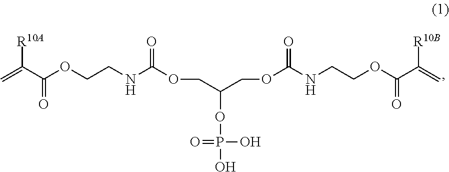

### (MMD 404)

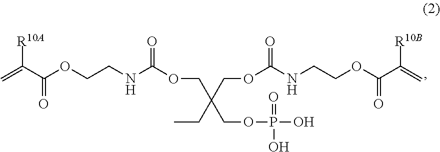

in particular

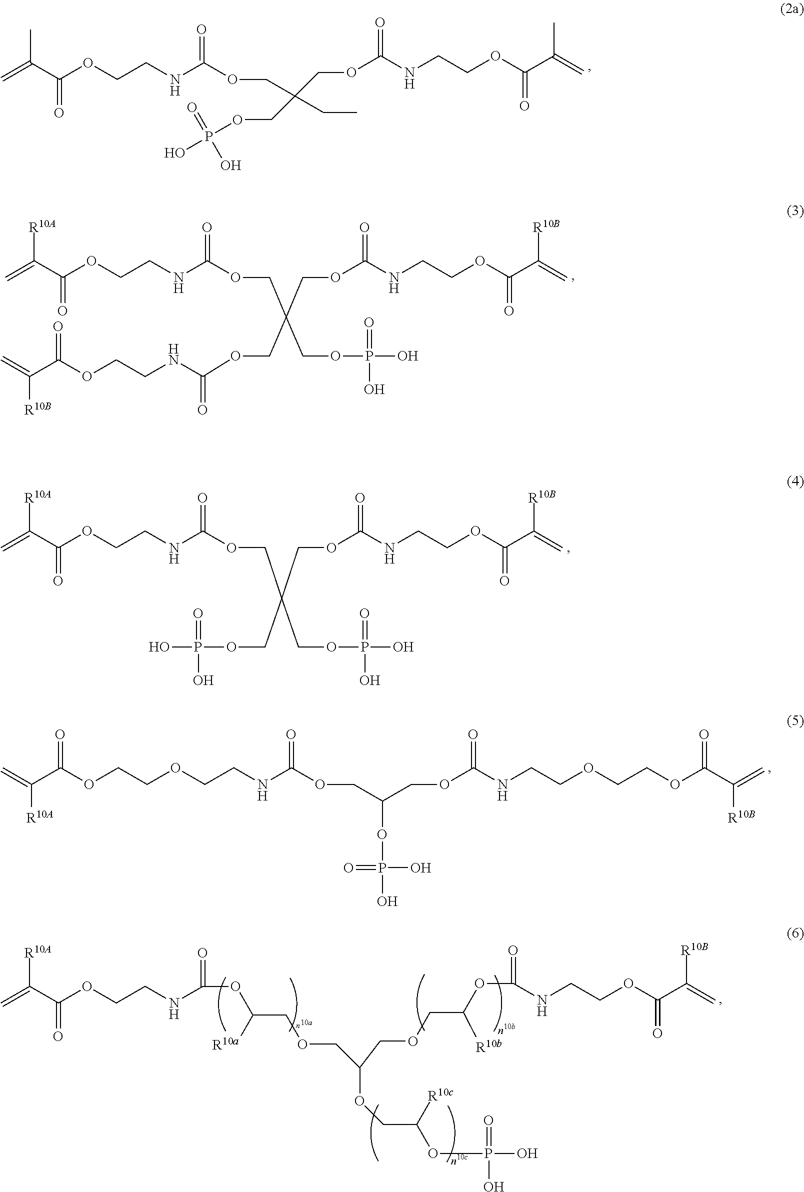

### (MMD406)

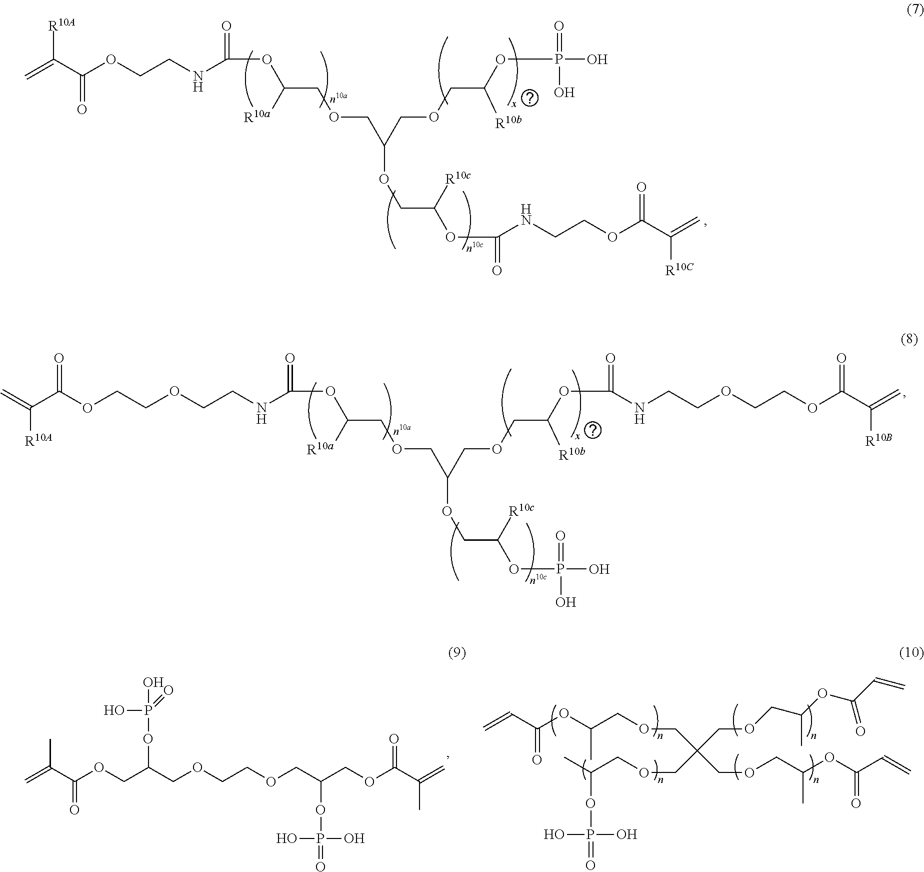

in formula (10) preferably n=1 to 10, especially preferably 1

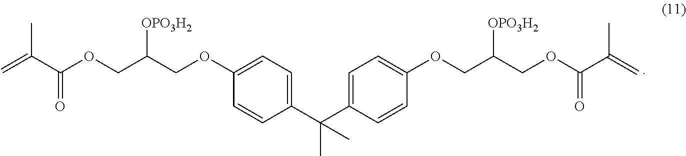

Particularly preferred acidic monomers comprise a c) mixture of acidic monomers comprises
i) at least one acidic monoester of at least one phosphoric acid of the general formulas I and/or II, 2-(meth)acryloyloxyethylphenylic acid phosphate and/or 10-(meth)-acryloyloxydecylic acid phosphate, and
(ii) 4-methacryloyloxyethyltrimellitic acid (4-MET) and/or its anhydride (4-META).

Preferably, the dental material in A) of the monomer component as c) i) comprises 4 to 20 wt.- % of at least one olefinic acid ester of a phosphoric acid and/or at least one olefinic acid ester of a thiophosphoric acid, in particular at least one acid monoester of at least one phosphoric acid of the general formulas I, Ia, Ib and/or II, 2-(meth)acryloyloxyethylphenylic acid phosphate and/or 10-(meth)acryloyloxydecylic acid phosphate, or mixtures of at least two of the acid esters, and
ii) 2 to 10 wt.- % of at least one olefinic carboxylic acid and/or at least one olefinic carboxylic anhydride, in particular
4-methacryloyloxyethyltrimellitic acid (4-MET) and/or its anhydride (4-META), with respect to the total composition of 100 wt.- % of the A) monomer component.

Preferably, the dental material comprises as c) i) 2 to 10 wt.- % of at least one olefinic acidic ester of a phosphoric acid and/or at least one olefinic acidic ester of a thiophosphoric acid, in particular at least one acidic monoester of at least one phosphoric acid of the general formulas I and/or II, 2-(meth)acryloyl oxyethylphenylic acid phosphate and/or 10-(meth)acryloyloxy-decylic acid phosphate, or mixtures of at least two of the acidic esters, and
ii) 1 to 5 wt.- % of at least one olefinic carboxylic acid and/or at least one olefinic carboxylic acid anhydride, in particular 4-methacryloyloxyethyltrimellitic acid (4-MET) and/or its anhydride (4-META), with respect to the total composition of 100 wt.- % of the dental material.

Another preferred dental material comprises, the A) monomer component optionally in admixture with B) the filler component, wherein the dental material comprises,
(a) 20 to 80 wt.- % of at least one at least difunctional urethane (meth)acrylate,
b) at least two hydrophilic alkylene oxide-based di-functional (meth)acrylate-based crosslinkers each selected from the combinations i) and ii) or i) and iii) or i), ii) and iii), with
i) 10 to 20 wt.- % 1,2-ethanediol dimethacrylate, diethylene glycol dimethacrylate (DEGMA), triethylene glycol dimethacrylate (TEGDMA), tetraethylene glycol dimethacrylate, propylene glycol dimeth acrylate, dipropylene glycol dimethacrylate, tripropylene glycol dimethacrylate, tetrapropylene glycol dimethacrylate, dipropylene glycol diacrylate, tripropylene glycol diacrylate, tetrapropylene glycol diacrylate, and/or
ii) 2 to 7.5 wt.- % of polyethylene glycol di(meth)acrylate and/or polypropylene glycol di(meth)acrylate or with at least five, in particular with five to twenty, ethylene glycol or propylene glycol groups, and mixtures thereof, and/or
iii) 2 to 7.5 wt.- % of 1,3-butylene glycol dimethacrylate, hexanediol dimethacrylate, octanediol dimethacrylate, decanediol dimethacrylate, dodecanediol dimethacrylate,
wherein the total content of i) and ii) or i) and iii) or i), ii) and iii) in the total composition is 12 to 25 wt.- %,
c) i) 4 to 20 wt.- % of acidic monoester of at least one phosphoric acid, in particular 9.5 to 20 wt.- %, preferably acidic monoester of at least one phosphoric acid of the general formulas I, Ia, Ib and/or II, 2-(meth)acryloyloxyethylphenyl acid phosphate and/or 10-(meth)acryloyloxydecylic acid phosphate,
ii) 2 to 10 wt.- %, in particular 3.75 to 10 wt.- %, of 4-(methacryloyloxyethyl)-trimellitic anhydride (4-META) and/or 4-methacryloyloxyethyltrimellitic acid (4-MET), and
d) 0.3 to 2.5 wt.- % of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe), and 0.6 to 2.5 wt.- % of at least one co-initiator selected from 2-n-butoxyethyl-4-(dimethylamino)benzoate and/or piperonyl alcohol, wherein the mass ratio of diketone to co-initiator is from 1:1.5 to 1:3, preferably 1:2 with +/−0.15,
e) 0 to 10 wt.- % of at least one tri-, tetra-, penta- and/or hexa-functional (meth)acrylate-based crosslinker which is not a urethane (meth)acrylate,
f) 0 to 5 wt.- % of at least one pigment, dye, UV stabilizer and/or vis stabilizer, wherein the total composition of monomer component A) is 100 wt.- %, and optionally
B) the filler component comprises
a) 90 to 100 wt.- % of at least one dental glass or a mixture of dental glasses,
b) 0 to 10 wt.- % of inorganic fluorides, metal oxide, mixed oxides, crystalline metal oxide and/or silicate, in particular 2 to 10 wt.- %, preferably 5 to 10 wt.- %,
c) 0 to 5 wt.- %, in particular 0.1 to 0.5 wt.- %, of at least one pigment, dye, UV and/or Vis stabilizer, and/or a mixture of at least two of the fillers,
wherein the total composition of filler component B) is 100 wt.- %, and,
wherein A) the monomer component and B) the filler component are present in the dental material in a mass ratio of 100:0 to 20:80, and wherein the total composition of the dental material is 100 wt.- %.

Further, according to one embodiment, a dental material is preferred comprising:
A) the monomer component in admixture with B) the filler component, said dental material comprising,
(a) 20 to 40 wt.- % of at least one at least difunctional urethane (meth)acrylate,
b) at least two hydrophilic alkylene oxide-based di-functional (meth)acrylate-based crosslinkers each selected from the combinations i) and ii) or i) and iii) or i), ii) and iii), with
i) 10 to 20 wt.- % 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, tetrapropylene glycol dimethacrylate dipropylene glycol diacrylate, tripropylene glycol diacrylate, tetrapropylene glycol diacrylate, and
ii) 2 to 7.5 wt.- % of polyethylene glycol di(meth)acrylate and/or polypropylene glycol di(meth)acrylate or with at least five, in particular with five to twenty, ethylene glycol or propylene glycol groups, and mixtures thereof, and/or
iii) 2 to 7.5 wt.- % of 1,3-butylene glycol dimethacrylate, hexanediol dimethacrylate, octanediol dimethacrylate, decanediol dimethacrylate, dodecanediol dimethacrylate,
wherein the total content of i) and ii) or i) and iii) or i), ii) and iii) in the total composition is 12 to 25 wt.- %,
c) i) 4 to 20 wt.- % of acidic monoester of at least one phosphoric acid, in particular 9.5 to 20 wt.- %, preferably acidic monoester of at least one phosphoric acid of the general formulas I, Ia, Ib and/or II, 2-(meth)acryloyloxyethylphenylic acid phosphate and/or 10-(meth)acryloyloxydecylic acid phosphate
ii) 2 to 10 wt.- %, in particular 3.75 to 10 wt.- %, of 4-(methacryloyloxyethyl)-trimellitic anhydride (4-META) and/or 4-methacryloyloxyethyltrimellitic acid (4-MET), and d) 0.3 to 2.5 wt.- % of at least one diketone selected from 1,7,7-trimethyl-bicyclo-[2.2.1]-hepta-2,3-dione (champherquinone) and/or phenylpropanedione, and
0.6 to 2.5 wt.- % of at least one co-initiator selected from 2-n-butoxyethyl-4-(dimethyl amino)benzoate and/or piperonyl alcohol, wherein the mass ratio of diketone to co-initiator is from 1:1.5 to 1:3, preferably 1:2 with +/−0.15,
e) 0 to 10 wt.- % of at least one tri-, tetra-, penta- and/or hexa-functional (meth)acrylate-based crosslinker which is not a urethane (meth)acrylate,
f) 0 to 5 wt.- %, in particular 0.1 to 0.5 wt.- %, of at least one pigment, dye, UV stabilizer and/or vis stabilizer, wherein the total composition of monomer component A) is 100 wt.- %, and optionally
B) the filler component comprises
a) 90 to 100 wt.- % of at least one dental glass or a mixture of dental glasses,
b) 0 to 10 wt.- % of inorganic fluorides, metal oxide, mixed oxides, crystalline metal oxide and/or silicate, in particular 2 to 10 wt.- %, preferably 5 to 10 wt.- %,
c) 0 to 5 wt.- % of at least one pigment, dye, UV and/or vis stabilizer, and/or a mixture of at least two of the fillers,
wherein the total composition of filler component B) is 100 wt.- %, and,
wherein A) the monomer component and B) the filler component are present in a mass ratio of 59:41 to 20:80 in the dental material, and wherein the total composition of the dental material is 100 wt.- %.

According to a preferred embodiment, a dental material, in particular a composite material, is comprised of a
A) Monomer component comprising
a) from 40 to 80 wt.- %, in particular from 50 to 80 wt.- %, of at least one at least difunctional urethane (meth)acrylate,
b) 10 to 35 wt.- %, in particular 15 to 35 wt.- %, preferably 20 wt.- % with +/−5 wt.- %, in particular with +/−3 wt.- %, of at least one hydrophilic alkylene oxide-based di-functional (meth)acrylate-based crosslinker which i) does not comprise urethane (meth)acrylate and/or
ii) no aromatic residues, or a mixture of at least two of these crosslinkers,
c) 5 to 30 wt.- %, in particular 10 to 20 wt.- %, of a mixture of acidic monomers as disclosed,
d) 0.3 to 5 wt.- %, in particular 0.3 to 3 wt.- %, of at least one initiator and/or initiator system, in each case having an absorption maximum of 420 to 550 nm, the content of the at least one diketone being 0.3 wt.- % in the monomer component, and optionally with at least one tert-amine as co-initiator or a 1,2-methylenedioxybenzene as co-initiator,
e) 0 to 10 wt.- %, in particular 0.01 to 5 wt.- %, of at least one tri-, tetra-, penta- and/or hexa-functional (meth)acrylate-based crosslinker which is not a urethane (meth)acrylate,
f) 0 to 5 wt.- %, in particular 0.1 to 0.6 wt.- % of at least one pigment, dye, UV stabilizer and/or vis stabilizer,
the total composition of monomer component A) being 100 wt.- %, and
with a B) filler component comprising
a) 90 to 100 wt.- % of at least one dental glass or a mixture of dental glasses,
b) 0 to 10 wt.- %, in particular 5 to 10 wt.- %, of at least one inorganic fluoride, preferably ytterbium fluoride, metal oxide, such as SiO₂, ZrO₂, and mixed oxides, in particular mixed oxides of SiO₂ and ZrO₂,
c) 0 to 5 wt.- %, in particular 0.1 to 0.6 wt.- %, of at least one pigment, dye, UV stabilizer and/or vis stabilizer,
wherein A) the monomer component and B) the filler component are present in a mass ratio of 60:40 to 40:60 in the composite material, and wherein the total composition of the composite material is 100 wt.- %.

Also an object of the invention is a dental material comprising
A) the monomer component optionally in admixture with B) the filler component, wherein the dental material comprises
a) 20 to 80 wt.- % of at least one at least difunctional urethane (meth)acrylate,
b) 8 to 20 wt.- % of at least one hydrophilic alkylene oxide-based di-functional (meth)acrylate-based crosslinker comprising i) no urethane (meth)acrylate and/or ii) no aromatic residues, or a mixture of at least two of these crosslinkers,
c) i) 4 to 20 wt.- % of at least one acidic monoester of at least one phosphoric acid of the general formulas I and/or II, 2-(meth)acryloyloxyethylphenylic acid phosphate and/or 10-(meth)-acryloyloxydecylic acid phosphate, and
ii) 1 to 10 wt.- %, in particular 1.5 to 5 wt.- %, of at least one olefinic carboxylic acid and/or at least one olefinic carboxylic anhydride, in particular at least one carboxylic acid-functionalized and/or carboxylic anhydride-functionalized (meth)acrylate monomer, and/or a mixture of i) and ii),
d) 0.3 to 2.5 wt.- %, in particular 0.4 to 1.5 wt.- %, of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe), and 0.6 to 2.5 wt.- %, in particular 1.0 to 2.0 wt.- %, of at least one co-initiator selected from tert-amine and 1,2-methylenedioxybenzenes, in particular selected from 2-n-butoxyethyl-4-(dimethylamino)benzoate and/or piperonyl alcohol, wherein the mass ratio of diketone to co-initiator selected from tert-amine or 1,2-methylenedioxybenzene is from 1:1.5 to 1:3,
e) 0 to 1 wt.- % of at least one pigment comprising titanium dioxide, and 0 to 2 wt.- % of dye, UV stabilizer and/or vis stabilizer, in particular 0.1 to 0.5 wt.- %, and
f) 0 to 76 wt.- %, in particular a) 0 to 40 wt.- % or b) 50 to 76 wt.- % of at least one dental glass or a mixture of dental glasses, in particular 50 to 76 wt.- %,
g) 0 to 10 wt.- % of inorganic fluoride, such as ytterbium fluoride, metal oxide, mixed oxides and/or silicates, in particular 2 to 10 wt.- %, preferably 5 to 10 wt.- %,
h) and 0 to 2 wt.- % of dye, UV and/or Vis stabilizer, and/or mixtures of at least two of the fillers e) to h), the above components a) to h) giving the total composition of 100 wt.- % of the dental material.

Another preferred dental material comprises, A) the monomer component optionally in admixture with B) the filler component, wherein the dental material comprises,
a) 20 to 30 wt.- % of at least one at least difunctional urethane (meth)acrylate,
b) at least two hydrophilic alkylene oxide-based di-functional (meth)acrylate-based crosslinkers each selected from the combinations i) and ii) or i) and iii) or i), ii) and iii), with
i) 5 to 10 wt.- % 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, tetrapropylene glycol dimethacrylate, dipropylene glycol diacrylate, tripropylene glycol diacrylate, tetrapropylene glycol diacrylate, and
ii) 1 to 5 wt.- % of polyethylene glycol di(meth)acrylate and/or polypropylene glycol di(meth)acrylate or with at least five, in particular with five to twenty, ethylene glycol or propylene glycol groups, and mixtures thereof and/or
iii) 1 to 5 wt.- % of 1,3-butylene glycol dimethacrylate, hexanediol dimethacrylate, octanediol dimethacrylate, decanediol dimethacrylate, dodecanediol dimethacrylate, wherein the total content of i) and ii) or i) and iii) or i), ii) and iii) in the total composition is 6 to 15 wt.- %,
c) i) 3 to 15 wt.- % of acidic monoester of at least one phosphoric acid, in particular 3 to 10 wt.- %, preferably acidic monoester, of at least one phosphoric acid of the general formulas I, Ia, Ib and/or II, 2-(meth)acryloyloxyethylphenylic acid phosphate and/or 10-(meth)acryloyloxy-decylic acid phosphate,
   ii) 0.5 to 5 wt.- %, in particular 3.75 to 10 wt.- % of 4-(methacryloyloxyethyl)
   trimellitic anhydride (4-META) and/or 4-methacryloyloxyethyltrimellitic acid (4-MET), and
d) 0.3 to 1.5 wt.- % of at least one diketone selected from 1,7,7-trimethyl-bicyclo-[2.2.1]-hepta-2,3-dione (champherquinone), phenylpropanedione, and 0.6 to 2.5 wt.- % of at least one co-initiator selected from 2-n-butoxyethyl-4-(dimethylamino)-benzoate and/or piperonyl alcohol, wherein the mass ratio of diketone to co-initiator is from 1:1.7 to 1:3, preferably 1:2 with +/−0.15,
e) 0 to 10 wt.- % of at least one tri-, tetra-, penta- and/or hexa-functional (meth)acrylate-based crosslinker which is not a urethane (meth)acrylate,
f) 0 to 5 wt.- %, in particular 0.1 to 0.5 wt.- %, of at least one pigment, dye, UV stabilizer and/or vis stabilizer,
g) 10 to 80 wt.- % of at least one dental glass or a mixture of dental glasses, in particular 50 to 80 wt.- %,
h) 0 to 10 wt.- % of inorganic fluorides, metal oxide, mixed oxides, crystalline metal oxide and/or silicate,
wherein the above components a) to h) give the total composition of 100 wt.- % of the dental material.

Preferably, the dental material, in particular a composite or fissure sealant, comprises in the monomer component at least a) urethane dimethacrylate (UDMA) and at least one monomer selected from i) TEGDMA, and ii) PEGDA (polyethylene glycol diacrylate) with n=2 to 20, PPGDA (polypropylene glycol diacrylate) with n=2 to 20, PEGDMA (polyethylene glycol dimethacrylate) with n=2 to 20, PPGDMA (polypropylene glycol dimethacrylate) with n=2 to 20, or DDDMA (dodecanediol dimethacrylate), and iii) BDMA, 1,4-butanediol dimethacrylate (1,4-BDMA) or pentaerythritol tetraacrylate, and optionally bis-GMA monomer (bisphenyl A-glycidyl methacrylate). In ii), n is preferably n=10 to 20.

Particularly preferably, the dental material, in particular a composite or fissure sealant, comprises in the monomer component at least: a) urethane dimethacrylate (UDMA) and at least two different monomers of the combination i) and ii) or i) and iii) or i), ii) and iii) with b) i) TEGDMA, and ii) PEGDA (polyethylene glycol diacrylate) with n=5 to 20, PPGDA (polypropylene glycol diacrylate) with n=5 to 20, PEGDMA (polyethylene glycol dimethacrylate) with n=5 to 20, PPGDMA (polypropylene glycol dimethacrylate) with n=5 to 20 or DDDMA (dodecanediol dimethacrylate), and iii) BDMA, 1,4-butanediol dimethacrylate (1,4-BDMA) or pentaerythritol tetraacrylate. Preferably, n=8 to 20, more preferably 10 to 20.

Further preferably, the dental material comprises in the monomer component at least: a) at least one difunctional urethane (meth)acrylate, preferably urethane dimethacrylate (UDMA), and at least one monomer, preferably at least two different monomers, of the combination i) and ii) or i) and iii) or i), ii) and iii) with b) i) TEGDMA, and ii) PEGDA (polyethylene glycol diacrylate) with n=10 to 20, PPGDA (polypropylene glycol diacrylate) with n=9 to 20, PEGDMA (polyethylene glycol dimethacrylate) with n=10 to 20, PPGDMA (polypropylene glycol dimethacrylate) with n=9 to 20, or DDDMA (dodecanediol dimethacrylate), and iii) BDMA, 1,4-butanediol dimethacrylate (1,4-BDMA) or pentaerythritol tetraacrylate, and optionally bis-GMA monomer (bisphenyl A-glycidyl methacrylate).

In monomer component A), the a) at least one at least difunctional urethane (meth)acrylate is preferably selected from difunctional urethane (meth)acrylate with a divalent alkylene group comprising difunctional urethane (meth)acrylates with a linear or branched divalent alkylene group having 3 to 20 carbon atoms, urethane dimethacrylate functionalized ethers with a linear or branched divalent alkylene group having 3 to 20 carbon atoms, urethane dimethacrylate functionalized polyethers with linear or branched bivalent alkylene group with 3 to 20 C atoms, urethane di-acrylate oligomer, bis(methacryloxy-2-ethoxycarbonylamino)alkylene and/or bis(methacryloxy-2-ethoxycarbonylamino)-substituted alkylene ethers are preferred, 1,6-bis(methacryloxy-2-ethoxycarbonylamino)-2,4,4-trimethylhexane (UDMA) is particularly preferred.

The difunctional urethane (meth)acrylate with bivalent alkylene group is preferably selected from linear or branched urethane dimethacrylates functionalized with a bivalent alkylene group, urethane dimethacrylate functionalized ethers or polyethers with alkylene group(s), such as bis(methacryloxy-2-ethoxycarbonylamino) alkylene, bis(meth-acryloxy-2-ethoxycarbonyl-amino) substituted polyalkylene ethers, preferably 1,6-bis(methacryloxy-2-ethoxycarbonyl-amino)-2,4,4-trimethylhexane, UDMA. Preferred is a bis(methacryloxy-2-ethoxycarbonyl-amino)alkylene, wherein alkylene comprises linear or branched C3 to C20, preferably C3 to C6, as particularly preferred an alkylene substituted with methyl groups. The divalent alkylene preferably comprises 2,2,4-trimethylhexamethylene and/or 2,4,4-trimethylhexamethylene. UDMA (1,6-bis(methacryloxy-2-ethoxycarbonylamino)-2,4,4-trimethylhexane) is particularly preferred.

Also an object of the invention is a dental material, in particular a composite material, which preferably comprises as
b) at least two different hydrophilic alkylene oxide-based di-functional (meth)acrylate-based crosslinkers, wherein one crosslinker is selected from i) and one crosslinker is selected from ii), or the crosslinker is in each case separately selected from i) and iii), or in each case at least one crosslinker is separately selected from i), ii) and iii), particularly preferably b) at least two different at least di-, tri-, tetra-, poly-functional alkylene oxide-based di-functional (meth)acrylate-based crosslinkers selected from:
(i) 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, Tetrapropylene glycol dimethacrylate dipropylene glycol diacrylate, tripropylene glycol diacrylate, tetrapropylene glycol diacrylate, 1,2-ethanediol diacrylate, diethylene glycol diacrylate (DEGA), triethylene glycol diacrylate (TEGDA), tetraethylene glycol diacrylate, propylene glycol diacrylate, dipropylene glycol dimethacrylate, and
ii) polyethylene glycol di(meth)acrylate and/or polypropylene glycol di(meth)acrylate or mixtures thereof, in particular with at least five ethylene glycol or propylene glycol groups, in particular polyethylene glycol di(meth)acrylates with n=5 to 20, in particular 5 to 15, ethylene glycol groups, n=5 to 12, preferably 5 to 9 alternatively from 10 to 15, ethylene glycol groups, and polypropylene glycol di(meth)acrylates with n=5 to n=20, in particular n=5 to 15, b n=5 to 12 preferably 5 to 9 alternatively from 10 to 15, linear or branched propylene glycol groups, pentapropylene glycol dimethacrylate, pentapropylene glycol diacrylate, hexapropylene glycol dimethacrylate, hexapropylene glycol diacrylate, octapropylene glycol dimethacrylate, octapropylene glycol diacrylate, nonapropylene glycol dimethacrylate, nonapropylene glycol diacrylate are preferred, decapropylene glycol dimethacrylate, decapropylene glycol diacrylate, undecapropylene glycol dimethacrylate, undecapropylene glycol diacrylate, dodecapropylene glycol dimethacrylate, dodecapropylene glycol diacrylate, and mixtures of at least two of the above-mentioned di(meth)acrylates, and/or
(iii) 1,3-butylene glycol dimethacrylate (butanediol dimethacrylate), hexanediol dimethacrylate, octanediol dimethacrylate, decanediol dimethacrylate, dodecanediol dimethacrylate.

Likewise, the composite material may comprise as: (e) at least one tri-, tetra-, penta- and/or hexa-functional (meth)acrylate-based crosslinker other than urethane (meth)acrylate and selected from (i) having three (meth)acrylate groups of trimethylolpropane trimethacrylate, ethoxylated-(15)-trimethylolpropane triacrylate, ethoxylated-5-pentaerythritol triacrylate, propoxylated-(5.5)-glyceryl triacrylate, trimethylolpropane triacrylate, tris(2-hydroxyethyl)isocyanurate triacrylate, and/or (ii) with four (meth)acrylate groups from di-trimethylolpropane tetraacrylate, ethoxylated-(4)-pentaerythritol tetraacrylate, pentaerythritol tetraacrylate, di-trimethylolpropane tetramethacrylate, ethoxylated-(4)-pentaerythritol tetramethacrylate, penta erythritol tetramethacrylate and/or (iii) with five (meth)acrylate groups from di-pentaerythritol pentaacrylate, i-pentaerythritol pentamethacrylate, dipentaerythritol pentaacrylate, di(tetra methylol methane) pentamethacrylate and/or (iv) with six (meth)acrylate groups a dipentaerythritol hexa(meth)acrylate.

The (c) mixture of acidic monomers preferably comprises.
i) at least one olefinic acid monoester of a phosphoric acid and/or at least one olefinic acid monoester of a thiophosphoric acid, in particular at least one phosphoric acid-based (meth)acrylate, phosphoric acid-based urethane (meth)acrylate and/or at least one thiophosphoric acid-based (meth)acrylate, comprising 2-(meth)acryloyloxyethyl acid phosphate, 2- and 3-(meth)acryloyloxypropyl acid phosphate, 4-(Meth)acryloyloxybutylic acid phosphate, 6-(Meth)acryloyloxyhexyl acid phosphate, 8-(meth)acryloyloxy octyl acid phosphate, 10-(meth)acryloyloxydecylic acid phosphate, 12-(meth)acryloyloxydodecylic acid phosphate, bis(2-(meth)acryloyloxyethyl acid phosphate, bis-2 or 3-(meth)acryloyloxypropylic acid phosphate, 2-(meth)acryloyloxyethylphenylic acid phosphate, 2-(meth)acryloyloxyethyl-p-methoxyphenylic acid phosphate, and the corresponding thiophosphonates of the aforementioned (meth)acrylates, preferably in combination with
ii) at least one olefinic carboxylic acid and/or at least one olefinic carboxylic anhydride comprising, in particular selected from, maleic acid, p-vinylbenzoic acid, 11-(meth)acryloyloxy-1,1-undecanedicarboxylic acid (MAC-10), 1,4-Di(meth)acryloyloxyethylpyromellitic acid, 6-(meth)acryloyloxyethylnaphthalene-1,2,6-tri carboxylic acid, 4-(meth)acryloyloxymethyltrimellit acid and anhydrides thereof, 4-(Meth)acryl-oyloxyethyltrimellitic acid and anhydride thereof, 4-(Meth)acryloyloxybutyltrimellitic acid and anhydride thereof, 4-[2-hydroxy-3-(meth)acryloyloxy] butyltrimellitic acid and anhydride thereof, 2,3-bis(3,4-dicarboxybenzoyloxy)propyl(meth)acrylate, 2-, 3-, or 4-(meth)acryloyloxy benzoic acid, N—O-di(meth)acryloyloxytyrosine, O-(meth)acryloyl oxy tyrosine, N-(meth)acryloyloxytyrosine, N-(meth)acryloyloxyphenylalanine, N-(meth)acryloyl-p-aminebenzoic acid, N-(meth)acryloyl-O-aminebenzoic acid, adduct of glycidyl (meth)acrylate with N-phenylglycine or N-tolylglycine, 4-[(2-hydroxy-3-(meth)acryloyloxypropyl)amino]phthalic acid, 3- or 4-[N-methyl-N-(2-hydroxy-3-(meth)acryloyloxypropyl)amino]phthalic acid, (meth)acryloyl aminosalicylic acid, and (meth)acryloyloxysalicylic acid, olefinic carboxylic acid having at least two carboxyl groups and/or olefinic carboxylic anhydride having at least three carboxyl groups, in particular selected from dicarboxylic acids, tricarboxylic acids, tetracarboxylic acids, addition product of 2-hydroxyethyl (meth)acrylate and pyromellite dianhydride (PMDM), an addition reaction product of 2 moles hydroxyethyl (meth)acrylate and 1 mole maleic anhydride or 3,3′,4,4′-benzophenonetetracarboxylic acid dianhydride (BTDA). Or 3,3′,4,4′-biphenyltetra-carboxylic acid dianhydride, and 2-(3,4-dicarboxybenzoyloxy) 1,3-di(meth)acryloyloxypropane.

In each case, the respective (meth)acrylates are independently selected from methacrylates and arcylates, and in particular this also applies to urethane (meth)acrylates, which are independently selected from urethamethacrylates and urethane acrylates, and in monoesters of phosphoric acid and all other olefinic compounds mentioned.

Particularly preferred are 11-(meth)acryloyloxy-1,1-undecanedicarboxylic acid (MAC-10) and 4-methacryloyloxyethyltrimellitic acid (4-MET) and/or its anhydride (4-META), and 2-(meth)acryloyloxyethylphenylic acid phosphate and/or 10-(meth)acryloyloxydecylic acid phosphate.

The content of the acidic monomers MDP and 4-META and/or 4-MET in the monomer component can be very low and very good adhesion values can be achieved with the composite according to the invention. For example, the content of MDP can be about 4 to 5 wt.- % in the monomer component and the content of 4-META and/or 4-MET can be about 2 wt.- % in the monomer component, and preferably an adhesion to dentin (bovine tooth) in the range of 16 to 21 MPa and to enamel (bovine tooth) in the range of 4 to 21 MPA can be measured.

In preferred alternatives, the composite material in the A) monomer component may comprise g) disubstituted 4,4′-di(oxabenzene)dialkylmethane of formula IX

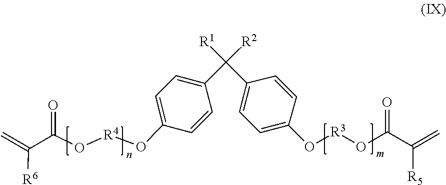

with R¹, R², R⁵ and R⁶ each in formula IX independently selected from H or C1 to C4 alkyl, and with R³ and R⁴ each linear, divalent C1 to C4 alkylene, with n=1 to 20 and m=1 to 20, preferably n=1 to 15 and m=1 to 15, in particular with n=3 and m=3 or n=10 and m=10 in formula IX. Particularly preferred are n+m=10 or n+m=3.

In further alternatives, the composite material in the A) monomer component may comprise. i) at least one monofunctional (meth)acrylate-based monomer, in particular comprising 2-hydroxyethylmethyl acrylate (HEMA). Alternatively or additionally suitable are also methacrylates containing urethane bonds, such as 2-(meth)acryloyloxy-ethyl isocyanate. Other suitable monofunctional (meth)acrylate-based monomers include aromatic vinyl compounds, such as styrene and divinylbenzene, vinyl esters, such as vinyl acetate, aliphatic esters of (meth)acrylic acid, such as methyl (meth)acrylate, ethyl (meth)acrylate, propyl (meth)acrylate, butyl (meth)acrylate, pentyl (meth)acrylate, hexyl (meth)acrylate.

According to a preferred embodiment, a dental material has a
A) monomer component comprising
a) from 40 to 80 wt.- % of at least one at least difunctional urethane (meth)acrylate,
b) 10 to 35 wt.- % of at least one hydrophilic alkylene oxide-based di-functional (meth)acrylate-based crosslinker which i) does not comprise urethane (meth)acrylate and/or
ii) does not comprise aromatic residues, or a mixture of at least two of these crosslinkers,
c) 5 to 30 wt.- % of a mixture of acidic monomers comprising
   i) at least one olefinic acid ester of a phosphoric acid, in particular of the formula I, Ia, Ib and/or II, and/or at least one olefinic acid ester of a thiophosphoric acid, in combination with
   ii) at least one olefinic carboxylic acid and/or at least one olefinic carboxylic acid anhydride,
d) 0.3 to 5 wt.- % of at least one initiator and/or initiator system, in each case having an absorption maximum of 420 to 550 nm, the content of the at least one diketone being 0.3 wt.-%, in particular greater than or equal to 0.55 wt.- %, in the monomer component, and greater than or equal to 1.1 wt.- % of at least one tert-amine as a co-initiator or a 1,2-methylenedioxybenzene as co-initiator,
e) 0 to 10 wt.- % of at least one tri-, tetra-, penta- and/or hexa-functional (meth)acrylate-based crosslinker which is not a urethane (meth)acrylate,
f) 0 to 5 wt.- % of at least one pigment, dye, UV stabilizer and/or Vis stabilizer, and
g) 5 to 25 wt.- %, in particular 5 to 15 wt.- %, of disubstituted 4, 4′-di(oxabenzene)dialkylmethane of formula IX with R¹, R², R⁵ and R⁶ in formula IX each being independently selected from H or C1 to C4 alkyl and with R³ and R⁴ each linear bivalent C1 to C4 alkylene wherein n=1 to 15 and m=1 to 15, the total composition of the monomer component A) being 100 wt.- %. The A) monomer component and the B) filler component may be present in the dental material in a ratio of A) to B) of 100:0 to 20:80 or, alternatively, in a ratio of A) to B) of 100:0 to 30:70.

As B) filler component, the dental material selected from fissure sealant or composite material preferably comprises at least one a) dental glass or a mixture of dental glasses, and b) optionally comprising at least one inorganic fluoride and/or amorphous metal oxide, mixed oxide, crystalline metal oxide, silicate, wherein a) the at least one dental glass comprises aluminosilicate glasses or fluoroaluminosilicate glasses, barium aluminum silicate, barium aluminum boron fluorosilicate, strontium silicate, strontium borosilicate, lithium silicate and/or lithium aluminum silicate, ytterbium fluoride-containing dental glasses, and mixtures of at least two of the foregoing dental glasses, and optionally b) comprises ytterbium fluoride, amorphous spherical fillers based on oxides or mixed oxides, such as amorphous SiO₂, ZrO₂ or also mixed oxides of SiO₂ and ZrO₂, fumed or precipitated silicas, amorphous silicas, phyllosilicates, quartz, feldspar and mixtures of at least two of the fillers. The fillers may all preferably be silanized. Common silanizing agents include (meth)acryloyloxy propyltrimethoxysilane and (meth)acryloyloxy propyltriethoxysilane or (meth)acryloyloxymethyl trimethoxysilane and (meth)acryloyloxymethyltriethoxysilane.

According to a preferred embodiment, the dental material, in particular the dental composite material, comprises at least one dental glass and/or mixtures thereof, in particular a radiopaque dental glass or mixtures thereof, of a mean particle size d₅₀ of 0.2 to 5.0 μm, in particular with d₅₀ of 0.2 to 2.0 μm, preferably with an average particle size of 0.2 to 1.75 μm, in particular with d₅₀ of 1.0 μm optionally plus/minus 0.6 μm, preferably plus/minus 0.05 μm, and preferably with d₉₉ less than or equal to 10 μm. Particularly preferred is a mean particle size of d₅₀ of about 0.85 μm optionally plus/minus 0.1 μm, in particular plus/minus 0.05 μm, preferably in particular plus/minus 0.03 μm, and preferably with d₉₉ less than or equal to 10 μm. A particularly preferred dental glass comprises barium aluminum borosilicate glass, especially barium aluminum borofluorosilicate. Further, a barium aluminosilicate glass having a refractive index of n=1.52 to 1.55, preferably 1.53. A particularly preferred particle size distribution may be in the range of d₁₀ with greater than or equal to 0.1 μm to d₉₉ less than or equal to 5 μm, preferably with d₁₀ greater than or equal to 0.4 μm to dog less than 2.5 μm, and an average diameter d₅₀ of 0.8 to 1.00 μm.

Furthermore, it is an object of the invention to provide a dental material comprising a filler component comprising at least one dental glass comprising barium aluminum borosilicate glass, barium aluminum borofluorosilicate glass, in particular silanized, preferably functionalized with methacryloxypropyl groups, and optionally at least one non-agglomerated amorphous metal oxide of a primary particle size of 2 to 45 nm, wherein the amorphous metal oxide comprises precipitated silica, zirconia, mixed oxides or mixtures thereof, in particular the metal oxides are silanized.

As initiator or initiator component, preferably d) the diketone is a 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe), and the at least one co-initiator preferably comprises 2-n-butoxyethyl-4-(dimethylamino)benzoate and/or as 1,2-methylenedioxybenzene piperonyl alcohol (1,2-methylenedioxybenzene-4-methanol). Other suitable tertiary amines may include 2-(ethylhexyl)-4-(N,N-dimethylamino)benzoate, dimethylaminobenzoic acid ester, triethanolamines, N,N-3,5-,N,N-3,5-tetramethyl aniline, 4-(dimethylamino) phenylethyl alcohol, dimethylaminobenzoic acid ester, 4-(N,N-dimethyl amino) benzoic acid.

At least one pigment, such as titanium dioxide, a dye, and in particular at least one UV and/or Vis stabilizer comprising 2-hydroxy4-methoxybenzoic acid may be present in both the monomer component and/or the filler component.

The c) mixture of acidic monomers preferably comprises i) ii) at least one olefinic acidic ester of phosphoric acid, preferably a monoester of a phosphoric acid, and/or at least one olefinic acidic ester of a thiophosphoric acid, preferably a monoester of a thiophosphoric acid, particularly preferably at least one monoester of a phosphoric acid and a (meth)acrylate, a monoester of a phosphoric acid and a urethane (meth)acrylate and/or at least one monoester of a thiophosphoric acid and a (meth)acrylate, particularly preferred are esters of the formula I, Ia, Ib and/or II and mixtures containing these, and ii) at least one olefinic carboxylic acid and/or at least one olefinic carboxylic anhydride, preferably at least one carboxylic acid-functionalized and/or carboxylic anhydride-functionalized (meth)acrylate monomer, i) and ii) being present in a mass ratio of 1:3 to 1:1, preferably from 1:2.75 to 1:1.75, preferably from 1:2.25 to 1:1.75. The above definition that (meth)acrylate or urethane (meth)acrylate comprises both the acrylate or the methacrylate also applies herein.

In a particularly preferred embodiment, the dental material in the A) monomer component in c) comprises as i) 4 to 20 wt.- %, in particular 9.5 to 20 wt.- %, at least one olefinic acid ester of a phosphoric acid and/or at least one olefinic acid ester of a thiophosphoric acid, preferably at least one monoester of a phosphoric acid and a (meth)acrylate, monoester of a phosphoric acid and a urethane (meth)acrylate, at least one monoester of a thiophosphoric acid and of a (meth)acrylate and/or at least one monoester of a thiophosphoric acid and of a urethane (meth)acrylate, particularly preferred are esters of the formula I, Ia, Ib and/or II and mixtures containing these, and as ii) 2 to 10 wt.- %, in particular 3.75 to 10 wt.- %, of at least one olefinic carboxylic acid and/or at least one olefinic carboxylic anhydride, preferably at least one carboxylic acid-functionalized and/or carboxylic anhydride-functionalized (meth)acrylate monomer, with respect to the total composition of 100 wt.- % of the A) monomer component. Throughout, (meth)acrylate is used synonymously with methyl acrylate or acrylate, i.e., any (meth)acrylates are disclosed as methyl acrylates or acrylates, whether disclosed as crosslinkers, urethanes or monoesters of a phosphoric acid.

Furthermore, a dental material is preferably provided with A) monomer component comprising
a) 50 to 80 wt.- % of at least one at least difunctional urethane (meth)acrylate,
b) 10 to 35 wt.- %, in particular 15 to 35 wt.- %, preferably 20 wt.- % with +/−5 wt.- %, in particular 20 wt.- %+/−3 wt.- %, of at least one hydrophilic alkylene oxide-based di-functional (meth)acrylate-based crosslinker which i) does not comprise urethane (meth)acrylate and/or
ii) does not comprise aromatic residues, or a mixture of at least two of these crosslinkers,
c) i) 4 to 20 wt.- %, in particular 9.5 to 20 wt.- %, of at least one olefinic acid ester of a phosphoric acid and/or at least one olefinic acid ester of a thiophosphoric acid, in particular at least one monoester of a phosphoric acid and a (meth)acrylate, a monoester of a phosphoric acid and urethane (meth)acrylate and/or at least one monoester of a thiophosphoric acid and a (meth)acrylate, in combination with ii) 2 to 10 wt.- %, in particular 3.75 to 10 wt.- %, of at least one olefinic carboxylic acid and/or at least one olefinic carboxylic anhydride, in particular at least one carboxylic acid-functionalized and/or carboxylic anhydride-functionalized (meth)acrylate monomer in combination with
d) 0.3 to 2.5 wt.- %, in particular 0.5 to 2.5 wt.- %, of at least one diketone selected from 1,7,7-trimethyl-bicyclo-[2.2.1]-hepta-2,3-dione (champherquinone), phenyl propanedione, benzoyltrimethylgerman (BTMGe) and/or dibenzoyldiethylgerman (DBDEGe) and 0.6 to 2.5 wt.- % in particular 1.0 to 2.5 wt.- % of at least one co-initiator selected from tert-amine and 1,2-methylenedioxybenzenes, in particular selected from 2-n-butoxyethyl-4-(dimethylamino)benzoate and/or piperonyl alcohol (1,2-methylenedioxybenzene-4-methanol), the mass ratio of diketone to co-initiator selected from tert-amine and 1,2-methylenedioxybenzenes, preferably a tert-amine, being 1:1.5 to 1:3, preferably 1:2, the value 2 being provided with a variation of +/−0.15,
e) 0 to 10 wt.- %, in particular 0.01 to 5 wt.- %, of at least one tri-, tetra-, penta- and/or hexa-functional (meth)acrylate-based crosslinker which is not a urethane (meth)acrylate, f) 0 to 5 wt.- %, in particular 0.1 to 0.6 wt.- % of at least one pigment, dye, UV stabilizer and/or vis stabilizer, the total composition of monomer component A) being 100 wt.- %, and with B) a filler component comprising
a) 90 to 100 wt.- % of at least one dental glass or a mixture of dental glasses,
b) 0 to 10 wt.- %, in particular 5 to 10 wt.- % of inorganic fluoride, such as ytterbium fluoride, metal oxide, such as SiO₂, mixed oxides, silicates, quartz, feldspar and/or mixtures thereof,
c) 0 to 5 wt.- %, in particular 0.1 to 0.6 wt.- %, of at least one pigment, such as TiO₂, dye, UV and/or Vis stabilizer, and/or
mixtures of at least two of the fillers, the total composition of the filler component being 100 wt.- %, and
wherein A) the monomer component and B) the filler component are present in a mass ratio of 70:30 to 30:70, in particular from 60:40 to 40:60, in the dental material and, the total composition of the dental material being 100 wt.- %.

In a preferred alternative, A) the monomer component and B) the filler component are present in the dental material in a mass ratio of 55:45 to 35:65 and the total composition of the dental material is 100 wt.- %.

Another particularly preferred dental material, in particular composite material comprises as
A) a monomer component comprising
(a) 50 to 80 wt.- % of at least one at least difunctional urethane (meth)acrylate,
b) at least two hydrophilic alkylene oxide-based di-functional (meth)acrylate-based crosslinkers each selected from the combinations i) and ii) or i) and iii) or i), ii) and iii), with i) 10 to 20 wt.- % 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, Tetrapropylene glycol dimethacrylate dipropylene glycol diacrylate, tripropylene glycol diacrylate, tetrapropylene glycol diacrylate, 1,2-ethanediol diacrylate, diethylene glycol diacrylate, triethylene glycol diacrylate, tetraethylene glycol diacrylate, propylene glycol diacrylate, dipropylene glycol diacrylate, and
ii) 2 to 7.5 wt.- % of polyethylene glycol di(meth)acrylate and/or polypropylene glycol di(meth) acrylate or having at least five ethylene glycol or propylene glycol groups, and mixtures thereof and/or
iii) 2 to 7.5 wt.- % of 1,3-butylene glycol dimethacrylate, hexanediol dimethacrylate, octanediol dimethacrylate, decanediol dimethacrylate, dodecanediol dimethacrylate, the total content of i) and ii) or i) and iii) or i), ii) and iii), in monomer component A) being 17.5 to 25 wt.- %, in particular 15 to 35 wt.- % or 20 wt.- % with +/−3 wt.- %,
c) i) 4 to 20 wt.- %, in particular 9.5 to 20 wt.- %, of acidic monoesters of phosphoric acids comprising monoesters of the formulas I, Ia, Ib and/or II, 2-(meth)acryloyloxyethylphenylic acid phosphate and/or 10-(meth)acryloyloxydecylic acid phosphate, and
ii) 2 to 10 wt.- %, in particular 3.75 to 10 wt.- %, of 4-(methacryloyloxyethyl)-trimellitic anhydride (4-META) and/or 4-methacryloyloxyethyltrimellitic acid (4-MET), and
d) 0.5 to 2.5 wt.- %, in particular 0.5 to 2.5 wt.- %%, of at least one diketone selected from 1,7,7-trimethyl-bicyclo-[2.2.1]-hepta-2,3-dione (champherquinone) and/or phenyl propanedione, and optionally mixtures comprising at least two diketones, and
1.0 to 2.5 wt.- %, in particular 1.1 to 2.5 wt.- %, of at least one co-initiator selected from 2-n-butoxyethyl-4-(dimethylamino)benzoate and/or piperonyl alcohol (1,2-methylene dioxybenzene-4-methanol), the mass ratio of diketone to co-initiator being 1:1.5 to 1:3, preferably 1:2 with a variation of the value 2 by +/−0.15,
e) 0 to 5 wt.- %, of at least one tri-, tetra-, penta- and/or hexa-functional (meth)acrylate-based crosslinker which is not a urethane (meth)acrylate,
f) 0 to 5 wt.- %, in particular 0.1 to 0.6 wt.- %, of at least one pigment, such as TiO₂, dye, UV and/or Vis stabilizer, the total composition of monomer component A) being 100 wt.- %, and
B) a filler component comprising
a) 90 to 100 wt.- % of at least one dental glass,
b) 0 to 10 wt.- %, in particular 5 to 10 wt.- % of inorganic fluorides, such as ytterbium fluoride, metal oxide, such as SiO₂, mixed oxides, crystalline metal oxide, silicate,
c) 0 to 5 wt.- %, in particular 0.1 to 0.6 wt.- %, of at least one pigment, such as TiO₂, dye, UV and/or Vis stabilizer, and/or a mixture of at least two of the fillers, and wherein the total composition of the filler component is 100 wt.- %, and wherein A) the monomer component and
B) the filler component are present in the composite material in a mass ratio of 59:41 to 20:80, and wherein the total composition of the composite material is 100 wt.- %.

In the stated compositions in which the contents of the components are named, the contents named after in particular are to be understood as belonging together; the same applies in subgroups to the contents named therein as preferred. the disclosure is also to be understood in connection with the examples not exhaustively listed and is addressed to the chemist or materials scientist.

Also an object of the invention is a polymerized dental material obtainable by polymerization, in particular by visible light polymerization, in particular the polymerized dental material has a shear bond strength to bovine tooth dentin of greater than or equal to 10 MPa, preferably greater than or equal to 15 MPa, preferably greater than or equal to 17 MPa, particularly preferably greater than or equal to 20 MPa, and/or a shear bond strength to bovine tooth enamel greater than or equal to 12 MPa, preferably greater than or equal to 19 MPa, preferably greater than or equal to 22 MPa, particularly preferably greater than or equal to 23 MPa.

It is also an object of the invention to use a curable or polymerizable dental material for radiation-curing, self-adhesive bonding to dentin and/or enamel of tooth, in particular human or veterinary tooth. Radiation curing is preferably carried out by means of visible light (Vis radiation), preferably in the wavelength range from 420 to 500 nm, preferably at 485 nm. Suitable light sources are LED illuminants.

Also an object of the invention is a self-adhesive, radiation-curable dental material, in particular a fissure sealant or a dental composite material or self-adhesive, polymerized, in particular radiation-cured composite material for use in sealing fissures in enamel and/or dentin, and/or for use for radiation-curing, self-adhesive bonding to substrates comprising metallic substrates, ceramic substrates and/or hybrid materials comprising polymers, in particular non-precious metal, zirconium, composites, precious metal, to dentin and/or enamel of tooth, in particular human or veterinary tooth. Also an object of the invention is a dental material for use as a self-adhesive fissure sealant or for use as a self-adhesive dental composite.

The following examples are intended to explain the subject matter of the invention without limiting it to the specific examples.

### Method Description:

Shear bond strength was measured according to DIN EN:ISO 29022 (2013). Bovine tooth enamel and/or dentin surfaces were prepared on SiC paper of grit size 120 to grit size 320. In the case of “Uncut” surfaces, bovine tooth enamel surfaces mechanically cleaned are used as bonding surfaces (Sof-Lex discs from 3M). The fissure sealant is massaged onto the tooth substrate and exposed to a Tranzlux Wave (Kulzer GmbH, spectrum: 440 to 480 nm, power density 1200 milliwatts/cm²) for 10 s. The material is then filled into cylindrical plastic molds (Ultradent equipment) and cured for 20 s. To simulate aging, some samples are subjected to thermal cycling (TWC=thermocyclic, thermal cycling/thermal cycling load for artificial aging) (5° C. to 55° C. water bath, 30 s dwell time and 5 s transfer time, 5,000 cycles). Shear bond strength is determined with a universal tester (crosshead speed 1 mm/min). 24 h measurements 37° C. in H₂ O. Measurement universal tester (room temperature). Unless otherwise stated, BTE/BTD are the 24 h measurements.

Flexural strength and flexural modulus of elasticity are performed according to the DIN-ISO 4049 standard (Polymer-based filling, restorative and luting materials, measurements at room temperature, 2019).

The determination of water absorption/solubility is carried out according to DIN-ISO 4049 section 7.12 to 4-8 Test specimens with 15 mm diameter and 1 mm width are cured, dried, stored for 7 days at 37° C. in H₂O and dried back.

The film thickness of the fissure sealant is determined according to ISO 4049 7.5. The material is loaded between 2 glass plates with 150 N for 180 s, cured and the difference measured with an outside micrometer.

The hardness is determined as follows: Cure test specimen 10 mm Delrinform/Teflonform for 20 s on the upper side (OS). Mark bottom side (US). Store for 24 h at 37° C. in H₂ O, grind flat and determine the penetration force (N/mm²)/penetration depth of the top side/bottom side with Zwick Universal Hardness using diamond.

The alkylene oxide-based di-functional methacrylates used in the following examples, such as TEGDMA, PEGDMA or DDDMA (dodecane diol dimethacrylate), particularly in preferred alternatives due to the combination of these and/or due to the content in the non-polymerized state of the fissure sealant, cause good flow to the enamel as well as penetration into fissures and thus the high bonding performance after radiation curing. Adhesion to the enamel is achieved without prior etching of the enamel.

Especially the concentration of the initiator as well as the ratios of diketone to co-initiator in the dental material are crucial, since a high conversion of the crosslinking during polymerization also results in good adhesion to the substrate, here dentin and/or enamel. The content of the combination of camphorquinone (CQ)/tertiary amine (2-n-butoxyethyl)-4-(dimethylamino)-benzoate, BEDB) should be greater than 0.2 wt.- %, in particular greater than 0.3 wt.- % for the diketone and greater than 0.4 wt.- %, in particular 0.6 wt.- % of the co-initiator in the initiator system in the monomer component, preferably in the total composition of the dental material. Table 1 shows the shear bond strength values obtained on ground bovine tooth enamel (BTE), uncut enamel (BTE-unground/BTE-uncut) after thermocycling (TWL) in otherwise qualitatively and quantitatively identically composed dental material (measurement according to ISO 29022 in MPa). The shear bond strength could be significantly increased with the aforementioned initiator system and with increased concentration of uncut BTE even after thermocycling (TWL).

Particle sizes of the fillers: The particle sizes are determined by laser diffraction. As a rule, particle size determination is carried out by laser diffraction with the Cilas instrument or alternatively Horiba LA-950 (Retsch) or DT1200 (Dispersion Technology). Unsilanized fillers are measured in water (dest) and silanized fillers are measured in isopropanol. The accuracy of the particle size determination is preferably plus/minus 0.1 micrometer for fillers with a particle size of, for example, 0.4 micrometer or d₅₀=0.4 micrometer. The accuracy for particle sizes of 1.5 micrometers or D50 1.5 micrometers is also at least in this range.

List of abbreviations:

| Abbreviations | |
|---|---|
| in Ex. | Chem. designation |
| UDMA | 1,6-Bis(methacryloxy-2-ethoxycarbonylamino)- |
| | 2,4,4-trimethylhexane |
| DDDMA | Dodecanediol dimethacrylate |
| E3BADMA | Formula IX, n = 3 |
| E10BADMA | Formula IX, n = 10 |
| MDP | 10-methacryloyloxydecyl phosphate |
| MMD404 | Formula I with formula III and two IV |
| MMD406 | Formula I with formula IIIb/IIIa and two formulas |
| | V, n = 1-10 |
| CQ | Camphorquinone |
| BEDB | 2-n-butoxyethyl)-4-(dimethylamino)-benzoate |

The alkylene oxide-based di-functional methacrylates used in the following examples, such as TEGDMA, PEGDMA or DDDMA (dodecane diol dimethacrylate), particularly due to the combination of these and/or due to the content in the non-polymerized state of the composite, cause good adhesion to the polar substrate of the dentin and the enamel and thus the high adhesion after radiation curing.

The initiator concentration in the composite is also very decisive, since a high turnover of crosslinking during polymerization with visible light also results in good adhesion to the substrate, in this case dentin and enamel. The content of the camphorquinone (CQ)/tertiary amine combination should preferably be greater than 0.3 wt.- %/greater than 0.6 wt.- % in the total composition. Table 1 shows the shear bond strength (SBS) values obtained on bovine tooth dentine (BTD) and bovine tooth enamel (BTE).

TABLE 1
SBS bovine dentin in MPa with different acidic monomers
(MDP, MMD404, MMD406) these are pure monomer blends
without fillers (a) 74.1 wt.-%, UDMA, 15 wt.-%
TEGDMA, 10 wt.-% acid monomer, b) 74.1 wt.-% UDMA,
14.1% TEGDMA, 10 wt.-% acid monomer:

| Monomer component | MDP | MMD404 | MMD406 |
|---|---|---|---|
| a) | | | |
| 0.3 wt.-% 1,7,7-Trimethyl- | 10.2 [MPa] | 17.5 [MPa] | 13.4 [MPa] |
| bicyclo[2.2.1]hepta-2,3-dion/ | | | |
| 0.6 wt.-% 2-n-Butoxyethyl- | | | |
| 4-(dimethylamino)benzoate | | | |
| b) | | | |
| 0.6 wt.-% 1,7,7-Trimethyl- | 17.2 [MPa] | 23.6 [MPa] | 18.2 [MPa] |
| bicyclo[2.2.1]hepta-2,3-dion/ | | | |
| 1.2 wt.-% 2-n-Butoxyethyl- | | | |
| 4-(dimethylamino)benzoate | | | |

The additional use of 4-META (4-methacryloyloxyethytrimellitic anhydride) in combination with a monomer containing phosphoric acid, such as MDP (10-methacryloyloxydecyl dihydrogen phosphate) significantly increases dentin adhesion. Especially in combination with a crosslinker/plasticizer with long PEG groups (PEG600 DMA, n=13) or dodecanediol dimethacrylate (DDDMA), which are hydrophilic alkylene oxide-based di-functional (meth)acrylate-based crosslinkers. The highest shear bond strengths can be achieved with composites combining acidic monomers of carboxylic acids or carboxylic acid anhydrides with acidic esters and two different alkylene oxide-based di-functional methacrylates comprising TEGDMA and PEGDMA or TEGDMA and DDDA, see Table 2a/2b.

TABLE 2a
Monomer component (each with an initiator content of 0.6 wt.-% CQ and
1.2 wt.-% BEDB

| Monomer | Example 4 | Example 5 | Example 6 | Example 7 | Example 8 | Example 9 | Example 10 |
|---|---|---|---|---|---|---|---|
| component | (wt.-%) | (wt.-%) | (wt.-%) | (wt.-%) | (wt.-%) | (wt.-%) | (wt.-%) |
| UDMA | 74.1 | 69.1 | 69.1 | 65.1 | 69.1 | 65.1 | 59.1 |
| TEGDMA | 14.1 | 15.1 | 14.1 | 14.1 | 14.1 | 14.1 | 15.1 |
| MDP | 10 | 10 | 10 | 10 | 10 | 10 | 10 |
| 4-Meta | | 4 | | 4 | | 4 | 4 |
| PEGDMA, | | | 5 | 5 | | | 5 |
| n = 13 | | | | | | | |
| DDDMA | | | | | 5 | 5 | 5 |
| SBS BTD | 17.2 | 23.7 | 17.0 | 28.1 | 14.3 | 25.2 | 23.0 |
| [MPa] | | | | | | | |
| SBS BTE | 15.9 | 16.8 | 14.9 | 26.36 | 14.4 | 23.7 | 16.9 |
| [MPa] | | | | | | | |

TABLE 2b
Monomer component (each with an initiator
content of 0.6 wt.-% CQ and 1.2 wt.-% BEDB

| | Example | Example | Example | Example |
|---|---|---|---|---|
| | 11 | 12 | 13 | 14 |
| Monomer component | Weight % | Weight % | Weight % | Weight % |
| UDMA | 74.1 | 74.1 | 74.1 | 72.1 |
| TEGDMA n = 3 | 14.1 | 14.1 | 14.1 | 12.1 |
| MMD404 | 10 | | | |
| MDP | | 10 | | |
| MMD406 | | | 10 | 10 |
| 4-META | | | | 4 |
| CQ (champerquinone) | 0.6 | 0.6 | 0.6 | 0.6 |
| BEDB (2-n-butoxyethyl)- | 1.2 | 1.2 | 1.2 | 1.2 |
| 4-(dimethylamino)- | | | | |
| benzoate | | | | |
| Total: | 100 | 100 | 100.0 | 100 |
| BTE [MPa] TWL | 11.7 | 6.2 | 2.8 | 9.4 |
| BTD [MPa] TWL | 13.7 | 18.2 | 7.6 | 23 |

The additional use of 4-META (4-methacryloyloxyethytrimellitic acid anhydride) in combination with a monomer containing phosphoric acid, such as MDP (10-methacryloyloxy decyl dihydrogen phosphate) again significantly increases adhesion. Especially in combination with a crosslinker/plasticizer with a longer PEG group than in TEGDMA, such as hepta(propylene glycol) diacrylate (n=7), hepta(ethylene glycol) diacrylate, n=7, or dodecanediol dimethacrylate (DDDMA, n=12). High shear bond strengths can be achieved with dental materials combining acidic monomers of carboxylic acids or carboxylic acid anhydrides with acidic esters of a phosphoric acid and two different alkylene oxide-based difunctional methacrylates comprising TEGDMA and PEGDMA (polyethylene glycol dimethylacrylate), TEGDMA and polyethylene glycol diacrylate, or TEGDMA and DDDA. Alternatively, very high shear bond strengths can be achieved with fissure sealants with the combination of acidic monomers of carboxylic acids or carboxylic acid anhydrides with acidic esters of a phosphoric acid and two different alkylene oxide-based difunctional acrylates comprising polypropylene glycol diacrylates with n=3 to less than 15, in particular with n=3 to 9. The propylene glycol diacrylate-based fissure sealants also show reduced water absorption with simultaneously increased shear bond strength with and without TWC.

With a filler component content of 60 wt.- % in the dental material to produce a composite, Examples 15, 16 and 17 were created, which clearly differ from the competitor Constic and Vertise Flow in their bonding performance. In Example 15, a diketone content according to the invention was used in relation to the co-initiator, as well as propylene glycol dimethylacrylate with n=13 as hydrophilic crosslinker.

TABLE 3a
Results shear adhesion state of the art

| | BTE- | RZS melting | BTD |
|---|---|---|---|
| | Enamel | TWL (TC) | Dentine |
| Vertise Flow (Kerr) | 16.4 | 2.1 | 5.9 (12.1) |
| Constic (DMG) | 15 | 12.0 | 4.8 |
| Venus Diamond Flow (Kulzer) | 4.3 | | 1.3 |

TABLE 3b
Examples 15, 16 and 17 with a filler content of 60 wt.-%.

| | Example 15 | Example 16 | Example 17 |
|---|---|---|---|
| | Weight % | Weight % | Weight % |
| UDMA | 64.02 | 54.00 | 63.2 |
| TEGDMA | 15.08 | 14.96 | 15.0 |
| E10BADMA | — | — | 10 |
| MDP | 9.96 | — | 10 |
| MMD | — | 20.17 | |
| 4-Meta | 4.00 | 4.06 | |
| PEGDMA, n = 13 | 5.07 | 5.00 | |
| (PEG600DMA). | | | |
| CQ | 0.62 | 0.60 | 0.6 |
| BEDB | 1.25 | 1.21 | 1.2 |
| Monomer matrix: | 100 | 100 | 100 |
| Composite | | | |
| Monomer matrix | 40 | 40 | 40 |
| Barium aluminum borosilicate | 60 | 60 | 60 |
| glass, 1.5 μm, 2.3 wt.-% | | | |
| silanized | | | |
| Composite | 100 | 100 | 100 |

TABLE 3c
Shear bond strength according to ISO 29022 on bovine tooth enamel
(BTE) mainly visible after aging in thermal load (TWL):

| | BTD | BTD-TWL | BTE | BTE-TWL |
|---|---|---|---|---|
| | [MPa] | [MPa] | [MPa] | [MPa] |
| Variant 1 | 21.2 | 17.6 | 22.7 | 14.7 |
| Variant 2 | 18.2 | 16.9 | 23.8 | 16.5 |
| Variant 3 | 15.7 | 14.1 | 20.4 | 16 |
| Vertise Flow | 5.9 | | 16.4 | 2.1 |
| Constic | 4.8 | | 15 | 12 |

TABLE 3d
Dental material properties

| | Flexural | E- | Shrinkage | Water- |
|---|---|---|---|---|
| | strength | modulus | stress | recording |
| | MPa | MPa | MPa (24 h) | μg/mm³ |
| Variant 1 | 99 | 5207 | 9.2 | 45.9 |
| Variant 2 | 121 | 5895 | 9.3 | 41.3 |
| Variant 3 | 121 | 5547 | 9.0 | 33.4 |
| Vertise Flow | 94 | 5379 | 8.1 | 61.9 |
| Constic | 101 | 4498 | 8.2 | 46 |

TABLE 4
Examples 18 to 22

| | Exam- | Exam- | Exam- | Exam- | Exam- |
|---|---|---|---|---|---|
| Monomer component | ple 18 | ple 19 | ple 20 | ple 21 | ple 22 |
| UDMA | 64.8 | 64.12 | 54.02 | 54.07 | 59.15 |
| TEGDMA | 14.72 | 15.08 | 14.97 | 15.08 | 15 |
| ethoxylated | — | — | — | 9.99 | — |
| dimethacrylate | | | | | |
| Formula IX, | | | | | |
| n = 3, m = 3 | | | | | |
| MDP | 9.82 | 9.86 | 20.19 | 10.04 | 10.02 |
| 4-META | 3.93 | 4 | 4 | 4 | 4 |
| PEGDMA, n = 13, | 4.91 | 5.07 | 4.99 | 5.04 | 5 |
| PEG600DMA | | | | | |
| HEMA (hydroxyethyl | — | — | — | — | 5.04 |
| methacrylate) | | | | | |
| CQ (champerquinone) | 0.59 | 0.62 | 0.61 | 0.6 | 0.61 |
| BEDB (2-n-butoxyethyl)- | 1.18 | 1.25 | 1.22 | 1.19 | 1.19 |
| 4-(dimethylamino)- | | | | | |
| benzoate | | | | | |
| DTBHQ (di-tert- | 0.05 | — | — | — | — |
| butylhydroquinone) | | | | | |
| Composite (mixture A) | | | | | |
| and B) | | | | | |
| Monomer component | 38.46 | 40 | 40 | 40 | 40 |
| Filler component | 61.54 | 60 | 60 | 60 | 60 |
| Filler component | | | | | |
| Barium aluminum boron | 53.85 | 60 | 60 | 60 | 60 |
| fluorosilicate glass, | | | | | |
| 1.5 μm sil. 2.3 wt.-% | | | | | |
| Ytterbium fluoride | 7.69 | — | — | — | — |
| Total | 100 | 100 | 100 | 100 | 100 |
| BTE (MPa) | 12.8 | 22.2 | 17.6 | 15.4 | 17.6 |
| BTD (MPa) | 11 | 21.2 | 13.4 | 10.1 | 16.4 |

TABLE 5

| Monomer component | Example 23 |
|---|---|
| UDMA | 61.1 |
| TEGDMA n = 3 | 14.1 |
| MDP | 10 |
| 4-Meta | 8 |
| PEGDMA, n = 13 | 5 |
| DDDMA, n = 12 | |
| CQ (champerquinone) | 0.6 |
| BEDB (2-n-butoxyethyl)- | 1.2 |
| 4-(dimethylamino)- | |
| benzoate | |
| Total | 100 |
| SBS BTD [MPa] | 28.2 ± 5.2 |
| SBS BTE [MPa] | 16.8 ± 7.1 |

TABLE 6
Monomer component - All variants incl. 0.6 wt.-% CQ, 1.2 wt.-% BEDB

| | Example | Example | Example | Example | Example | Example |
|---|---|---|---|---|---|---|
| | 24 | 25 | 26 | 27 | 28 | 29 |
| | Weight % | Weight % | Weight % | Weight % | Weight % | Weight % |
| UDMA | 65.1 | 65.1 | 60.1 | 65.1 | 65.1 | 65.1 |
| TEGDMA | 14.1 | 9.1 | 14.1 | 9.1 | — | 9.1 |
| MDP | 10 | 10 | 10 | 10 | 10 | 10 |
| 4-METAa | 4 | 4 | 4 | 4 | 4 | 4 |
| PEGDMA | 5 | | | | | |
| PPGDMA, | | 10 | | | | |
| n = 7 | | | | | | |
| PPGDMA, | | | 10 | 10 | 19.1 | |
| n = 3 | | | | | | |
| PPGDMA, | | | | | | 10 |
| n = 12 | | | | | | |
| (Mw approx. 800) | | | | | | |
| BTE [MPa] | | | 23.8 ± 6.8 | 22.1 ± 4.8 | 25.6 ± 5.8 | |
| BTD [MPa] | 25.2 ± 4.0 | 16.1 ± 2.8 | 19.6 ± 2.3 | 22.3 ± 6.9 | 23.2 ± 4.9 | 17.7 ± 3.9 |

TABLE 7a
With 60 wt.-% filler for composite formulation with different
acidic monomer combinations, respectively EBADMA and HEMA.

| | Example | Example | Example | Example |
|---|---|---|---|---|
| | 30 | 31 | 32 | 33 |
| Monomer component | Weight % | Weight % | Weight % | Weight % |
| UDMA | 25.6 | 21.6 | 21.67 | 23.65 |
| TEGDMA, n = 3 | 6 | 6 | 6 | 6 |
| E3BADMA, n = 3 | — | — | 4 | — |
| Formula IX | | | | |
| MMD 404 | | | | |
| MDP | 4 | 8 | 4 | 4 |
| 4-Meta | 1.6 | 1.6 | 1.6 | 1.6 |
| PEGDMA, n = 13 | 2.05 | 2.05 | 2.05 | 2 |
| HEMA | — | — | — | 2 |
| CQ | 0.25 | 0.25 | 0.25 | 0.25 |
| BEDB | 0.5 | 0.5 | 0.5 | 0.5 |
| Barium aluminum boron | 60 | 60 | 60 | 60 |
| fluorosilicate glass, | | | | |
| 1.5 μm sil. 2.3 wt.-% | | | | |
| Total: | 100.0 | 100.0 | 100.1 | 100.0 |
| BTE (MPa) | 22.2 | 17.6 | 15.4 | 17.6 |
| BTD (MPa) | 21.2 | 13.4 | 10.1 | 16.4 |

TABLE 7b
With 60 wt.-% filler for composite formulation with different
acidic monomer combinations, respectively EBADMA and HEMA

| | Exam- | Exam- | Exam- | Exam- | Exam- |
|---|---|---|---|---|---|
| | ple 34 | ple 35 | ple 36 | ple 37 | ple 38 |
| Monomer component | wt.-% | wt.-% | wt.-% | wt.-% | wt.-% |
| UDMA | 21.6 | 19.58 | 23.68 | 25.28 | 23.28 |
| TEGDMA, n = 3 | 6 | 6 | 6 | 6 | 6 |
| E3BADMA, n = 3 | | | | | |
| Formula IX | | | | | |
| E10BADMA, n = 10 | | | | 4 | 4 |
| Formula IX | | | | | |
| MMD 404 | 8.08 | 8.1 | 2 | | 2 |
| MDP | | | 4 | 4 | 4 |
| 4-Meta | 1.6 | 1.6 | 1.6 | | |
| PEGDMA, n = 13 | 2 | 2 | 2 | | |
| HEMA | | 2 | | | |
| CQ | 0.24 | 0.24 | 0.24 | 0.24 | 0.24 |
| BEDB | 0.48 | 0.48 | 0.48 | 0.48 | 0.48 |
| Barium aluminum boron | 60 | 60 | 60 | 60 | 60 |
| fluorosilicate glass, | | | | | |
| 1.5 μm sil. 2.3 wt.-% | | | | | |
| Total: | 100.0 | 100.0 | 100.0 | 100.0 | 100.0 |
| BTE (MPa) | 14.8 | 21.5 | 23.8 | 20.4 | 25.2 |
| BTD (MPa) | 12.7 | 11.2 | 18.2 | 15.7 | 14.1 |

TABLE 8a
With 60 wt.-% filler as composite formulation with
various acidic monomer combinations, or TEGDMA
and further crosslinkers hydrophilic crosslinkers
or crosslinkers of formula IX, such as EBADMA

| | Example 39 | Example 40 | Example 41 |
|---|---|---|---|
| | Weight % | Weight % | Weight % |
| UDMA | 25.68 | 23.68 | 25.28 |
| TEGDMA, n = 3 | 6 | 6 | 6 |
| E3BADMA, n = 3 | | | |
| Formula IX | | | |
| E10BADMA, n = 10 | | | 4 |
| Formula IX | | | |
| PEGDMA, n = 13 | 2 | 2 | |
| DDMA, n = 12 | | | |
| MDP | 4 | 4 | 4 |
| 4-Meta | 1.6 | 1.6 | |
| MMD404 | | 2 | |
| CQ | 0.24 | 0.24 | 0.24 |
| BEDB | 0.48 | 0.48 | 0.48 |
| Barium aluminum boron | 60 | 60 | 60 |
| fluorosilicate glass, | | | |
| 1.5 μm sil. 2.3 wt.-% | | | |
| Total | 100 | 100 | 100 |
| Shear bond strength | 17.6 | 16.9 | 14.1 |
| BTD - TWL (MPa) | | | |
| Shear bond strength | 14.7 | 16.5 | 16 |
| BTE - TWL (MPa) | | | |
| Flexural strength | 99 | 121 | 121 |
| E-modulus | 5207 | 5895 | 5547 |
| Water absorption (μg/mm³) | 45.9 | 41.3 | 33.4 |
| Solubility (μg/mm³) | 0.5 | 1.4 | 0.9 |

TABLE 8b
With 60 wt.-% filler as composite formulation with
various acidic monomer combinations, or TEGDMA
and further hydrophilic crosslinkers as well as
crosslinkers of formula IX, such as EBADMA

| | Example 42 | Example 43 |
|---|---|---|
| | wt.-% | wt.-% |
| UDMA | 25.68 | 23.68 |
| TEGDMA n = 3 | 6 | 6 |
| E10BADMA n = 10 | | |
| Formula IX | | |
| PEGDMA n = 13 | | 2 |
| DDMA n = 12 | 2 | 2 |
| MDP | 4 | 4 |
| 4-Meta | 1.6 | 1.6 |
| MMD404 | | |
| CQ | 0.24 | 0.24 |
| BEDB | 0.48 | 0.48 |
| Barium aluminum boron | 60 | 60 |
| fluorosilicate glass, | | |
| 1.5 μm sil. 2.3 wt.-% | | |
| Total | 100 | 100 |
| Shear bond strength | 13.4 | 9.2 |
| BTD - TWL (MPa) | | |
| Shear bond strength | 13.2 | 15.8 |
| BTE - TWL (MPa) | | |

TABLE 8c
Comparative examples

| Products from the market: | Constic | Vertise Flow |
|---|---|---|
| Water absorption (μg/mm³) | 45.6 | 61.9 |
| Solubility (μg/mm³) | 3.8 | 8.6 |

TABLE 9
Dental material comprising monomers a), b)
and c) with variation in the filler system

| | Exam- | Exam- | Exam- | Exam- | Exam- |
|---|---|---|---|---|---|
| Filler Systems Matrix | ple 44 | ple 45 | ple 46 | ple 47 | ple 48 |
| UDMA | 25.68 | 25.68 | 25.68 | 25.68 | 25.68 |
| TEGDMA n = 3 | 6 | 6 | 6 | 6 | 6 |
| MDP | 4 | 4 | 4 | 4 | 4 |
| 4-META | 1.6 | 1.6 | 1.6 | 1.6 | 1.6 |
| PEG-DMA n = 13 | 2 | 2 | 2 | 2 | 2 |
| CQ | 0.24 | 0.24 | 0.24 | 0.24 | 0.24 |
| BEDB | 0.48 | 0.48 | 0.48 | 0.48 | 0.48 |
| Barium aluminum | 60 | 50 | 40 | 30 | |
| boron fluorosilicate | | | | | |
| glass, 1.5 μm sil. | | | | | |
| 2.3 wt.-% | | | | | |
| Barium aluminum | | 10 | 20 | 30 | |
| boron fluorosilicate | | | | | |
| glass, 0.4 μm sil. | | | | | |
| 5.6 wt.-% | | | | | |
| Barium aluminum | | | | | 60 |
| boron fluorosilicate | | | | | |
| glass, 0.4 μm, | | | | | |
| unsilanized | | | | | |
| Total | 100 | 100 | 100 | 100 | 100 |
| RTE | 22.7 | 25.3 | 15.5 | 18.2 | 20.5 |
| RTD | 21.2 | 17.6 | 13.0 | 12.0 | 20.9 |
| Flexural strength/ | 99/5207 | 122/6295 | | | |
| E-modulus | | | | | |

1. A self-adhesive, radiation curable, polymerizable dental material comprising
A) a monomer component comprising
a) at least one at least difunctional urethane (meth)acrylate, wherein in each case independently urethane (meth)acrylate is selected from urethane methacrylate and urethane acrylate,
b) at least one hydrophilic alkylene oxide-based di-functional (meth)acrylate-based crosslinker, which i) does not comprise urethane (meth)acrylate and/or ii) does not comprise aromatic residues, or a mixture of at least two of these crosslinkers, wherein in each case independently one (meth)acrylate is selected from methacrylate and acrylate,
c) at least one olefinic acid ester of a phosphoric acid and/or at least one olefinic acid ester of a thiophosphoric acid,
d) at least one initiator and/or initiator system each having an absorption maximum of 420 to 550 nm and comprising i) diketone and at least one ii) co-initiator selected from a tert-amine and a 1,2-methylenedioxybenzene; and
g) disubstituted 4,4′-di(oxabenzene)dialkylmethane of the formula IX 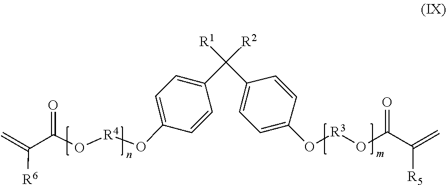
with R¹, R², R⁵ and R⁶ in Formula IX each independently selected from H or C1 to C4-alkyl, and with R³ and R⁴ each linear and divalent C1 to C4 alkylene with n=2 to 15 and m=2 to 15; and optional
B) a filler component comprising at least one dental glass or mixtures of dental glasses, wherein the total composition of the dental material is 100% by weight wherein
i) diketone and ii) co-initiator are present at a molar ratio of ii):i) of greater than or equal to 1.1, wherein i) the diketone is present at greater than or equal to 0.2% by weight in the total composition.

2. Dental material according to claim 1, which comprises
c) a mixture of acidic monomers comprising
i) at least one olefinic acid ester of a phosphoric acid and/or at least one olefinic acidic ester of a thiophosphoric acid, in combination with
ii) at least one olefinic carboxylic acid and/or at least one olefinic carboxylic acid anhydride.

3. Dental material according to claim 1, comprising
d) at least one initiator and/or initiator system comprising i) diketone and ii) co-initiator selected from a tert-amine having a molar ratio of ii):i) of greater than or equal to 1.15, and wherein i) the diketone is present at greater than or equal to 0.4% by weight in the total composition of the dental material of 100% by weight.

4. Dental material according to claim 1, which comprises
i) at least one olefinic acid ester of a phosphoric acid and/or at least one olefinic acid ester of a thiophosphoric acid comprising at least one monoester of a phosphoric acid and a (meth)acrylate, monoester of a phosphoric acid and a urethane (meth)acrylate and/or at least one monoester of a thiophosphoric acid and a (meth)acrylate, wherein in each case (meth)acrylate is independently selected from methacrylate and acrylate and urethane (meth)acrylate is independently selected from urethane methacrylate and urethane acrylate.

5. Dental material according to claim 1, which comprises
A) a monomer component comprising
a) from 40 to 80% by weight of at least one at least difunctional urethane (meth)acrylate,
b) 10 to 35% by weight of at least one hydrophilic alkylene oxide-based di-functional (meth)acrylate-based crosslinker which i) does not comprise urethane (meth)acrylate and/or ii) does not comprise aromatic residues, or a mixture of at least two of these crosslinkers,
c) 5 to 30% by weight of a mixture of acidic monomers comprising
i) at least one olefinic acid ester of a phosphoric acid and/or at least one olefinic acidic ester of a thiophosphoric acid, in combination with
ii) at least one olefinic carboxylic acid and/or at least one olefinic carboxylic acid anhydride,
d) 0.3 to 5% by weight of at least one initiator and/or initiator system, in each case having an absorption maximum of 420 to 550 nm, the content i) of the at least one diketone is greater than or equal to 0.3% by weight, and of ii) at least one co-initiator is greater than or equal to 0.6% by weight in the monomer component,
e) 0 to 10% by weight of at least one tri-, tetra-, penta- and/or hexa-functional (meth)-acrylate-based crosslinker which is not a urethane (meth)acrylate,
f) 0 to 5% by weight of at least one pigment, dye, UV stabilizer and/or Vis stabilizer, wherein the total composition of monomer component A) is 100% by weight, and optionally
B) a filler component comprising
a) 90 to 100% by weight of at least one dental glass or mixtures of dental glasses,
b) 0 to 10% by weight of inorganic fluorides, metal oxide, and/or mixed oxides,
c) 0 to 5% by weight of at least one pigment, dye, UV stabilizer and/or Vis stabilizer, the total composition of filler component B) being 100% by weight,
wherein A) the monomer component and B) the filler component are present in the dental material in a mass ratio of 100:0 to 20:80, and wherein the total composition of the dental material is 100% by weight.

6. Dental material according to claim 1, wherein
A) the monomer component and B) the filler component are present in the dental material in a mass ratio of 100:0 to 60:40, and wherein the total composition of the dental material is 100% by weight.

7. Dental material according to claim 1, wherein
the i) acidic monoesters comprises at least one phosphoric acid of the general formulae I, Ia, Ib and/or II or mixtures of at least two of the monoesters selected from formulae I, Ia, Ib and II 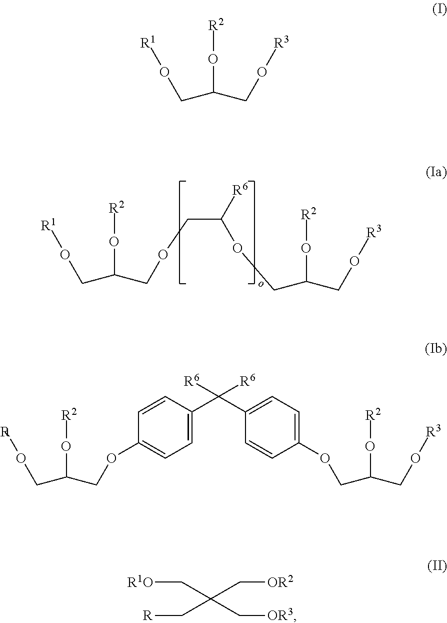
with R=CH₃, OR¹, OR² or OR³
wherein R¹, R² and R³ in formulas I, Ia, Ib and II are each independently selected from formulas III, IIIa, IIIb, IV, V, VIa, VIb, VIIa, VIIb, VIIIa and VIIIb wherein 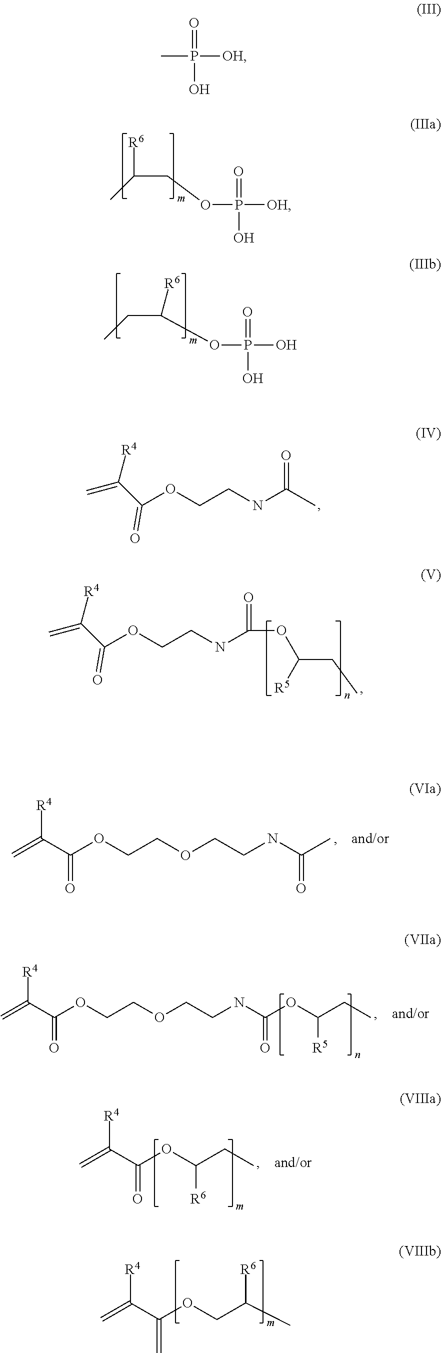
with o=1 to 100 in formula Ia,
wherein m=1 to 100 in formulas IIIa and/or IIIb, where in formulae IIIa and IIIb R⁶ is selected from H and methyl, and
wherein, in the formulas V and Vila, n is independently from 1 to 100, and
wherein formulas I, Ia, Ib and II at least one residue or two residues selected from R¹, R² and R³ correspond to a group of formula III or IIIa and the remaining residue or residues of R¹, R² and R³ are selected from at least one group of formulas IV, V, VIa, VIIa and/or VIIIb, wherein R⁴ and R⁵ are each independently selected in the groups of formulae IV, V, VIa, VIb, VIIa, VIIb, VIIIa and VIIIb from H, methyl and ethyl, and
wherein R⁶ is independently selected from H and methyl in each of the groups of formulae Ia, Ib, VIIIa and VIIIb,
where m=0 or 1 to 100 in formulas VIIIa and/or VIIIb, with R⁶ selected from H and methyl.

8. Dental material according to claim 1,
which comprises A) the monomer component optionally in admixture with B) the filler component, said dental material comprising,
a) 20 to 80% by weight of at least one at least difunctional urethane (meth)acrylate,
b) at least two hydrophilic alkylene oxide-based di-functional (meth)acrylate-based crosslinkers each selected from the combinations i) and ii) or i) and iii) or i), ii) and iii), with
i) 10 to 20 wt. % 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, 1,2-ethanediol diacrylate, diethylene glycol diacrylate, triethylene glycol diacrylate, tetraethylene glycol diacrylate, propylene glycol diacrylate, dipropylene glycol diacrylate, tripropylene glycol dimethacrylate, tetrapropylene glycol dimethacrylate, tripropylene glycol diacrylate, or tetrapropylene glycol diacrylate, and/or
ii) 2 to 7.5% by weight of polyethylene glycol di(meth)acrylate and/or polypropylene glycol di(meth)acrylate or with at least five, in particular with five to twenty, ethylene glycol or propylene glycol groups, and mixtures thereof, and/or
iii) 2 to 7.5% by weight of 1,3-butylene glycol dimethacrylate, hexanediol dimethacrylate, octanediol dimethacrylate, decanediol dimethacrylate, or dodecanediol dimethacrylate,
wherein the total content of i) and ii) or i) and iii) or i), ii) and iii) in the total composition is 12 to 25% by weight,
c) i) 4 to 20% by weight of acidic monoester of at least one phosphoric acid of the general formulae I, Ia, Ib and/or II, 2-(meth)acryloyloxyethylphenylic acid phosphate, and/or 10-(meth)acryloyloxydecyl acid phosphate,
ii) 2 to 10% by weight of 4-(methacryloyloxyethyl)trimellitic anhydride (4-META) and/or 4-methacryloyloxyethyltrimellitic acid (4-MET), and
d) 0.3 to 2.5% by weight of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe), and 0.6 to 2.5% by weight of at least one co-initiator selected from 2-n-butoxyethyl-4-(dimethylamino)benzoate and/or piperonyl alcohol, wherein the mass ratio of diketone to co-initiator is from 1:1.5 to 1:3,
e) 0 to 10% by weight of at least one tri-, tetra-, penta- and/or hexa-functional (meth)acrylate-based crosslinker which is not a urethane (meth)acrylate,
f) 0 to 5% by weight of at least one pigment, dye, UV stabilizer, and/or vis stabilizer,
wherein the total composition of monomer component A) is 100% by weight, and optionally
B) the filler component comprises
(a) 90 to 100% by weight of at least one dental glass or a mixture of dental glasses,
b) 0 to 10% by weight of inorganic fluorides, metal oxide, mixed oxides, crystalline metal oxide and/or silicate,
c) 0 to 5% by weight of at least one pigment, dye, UV and/or vis stabilizer, and/or a mixture of at least two of the fillers,
wherein the total composition of filler component B) is 100% by weight, and,
wherein A) the monomer component and B) the filler component are present in the dental material in a mass ratio of 100:0 to 20:80, and wherein the total composition of the dental material is 100% by weight.

9. Dental material according to claim 1,
which comprises A) the monomer component in admixture with B) the filler component, and wherein A) the monomer component and B) the filler component are present in the mass ratio of 59:41 to 20:80, and wherein the total composition of the dental material is 100% by weight.

10. Dental material according to claim 1, which comprises
a) at least one at least difunctional urethane (meth)acrylate selected from difunctional urethane (meth)acrylate having a divalent alkylene group comprising difunctional urethane (meth)acrylates having a linear or branched divalent alkylene group having 3 to 20 carbon atoms, urethane dimethacrylate functionalized ethers having a linear or branched bivalent alkylene group with 3 to 20 C atoms, or urethane dimethacrylate functionalized polyethers having a linear or branched bivalent alkylene group with 3 to 20 C atoms.

11. Dental material according to claim 1, which comprises as
b) at least one hydrophilic alkylene oxide-based di-functional (meth)acrylate-based crosslinker selected from
i) 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, tetrapropylene glycol dimethacrylate, dipropylene glycol diacrylate, tripropylene glycol diacrylate, or tetrapropylene glycol diacrylate, and
(ii) polyethylene glycol di(meth)acrylate and/or polypropylene glycol di(meth)acrylate or mixtures of these, and/or
(iii) 1,3-butylene glycol dimethacrylate (butanediol dimethacrylate), hexanediol dimethacrylate, octanediol dimethacrylate, decanediol dimethacrylate and/or dodecanediol dimethacrylate.

12. Dental material according to claim 1, which comprises
at least one hydrophilic alkylene oxide-based di-functional (meth)acrylate-based crosslinker selected from polyethylene glycol di(meth)acrylate and/or polypropylene glycol di(meth)acrylate or mixtures thereof.

13. Dental material according to claim 1, wherein
at least one hydrophilic alkylene oxide-based di-functional (meth)acrylate-based crosslinker is selected from
Polyethylene glycol di(meth)acrylate and/or polypropylene glycol di(meth)acrylate or mixtures thereof selected from pentapropylene glycol dimethacrylate, pentapropylene glycol diacrylate, hexapropylene glycol dimethacrylate, hexapropylene glycol diacrylate, octapropylene glycol dimethacrylate, octapropylene glycol diacrylate, nonapropylene glycol dimethacrylate, nonapropylene glycol diacrylate, decapropylene glycol dimethacrylate, decapropylene glycol diacrylate, undecapropylene glycol dimethacrylate, undecapropylene glycol diacrylate, dodecapropylene glycol dimethacrylate, dodecapropylene glycol diacrylate, and mixtures of at least two of the aforementioned di(meth)acrylates.

14. Dental material according to claim 1, which comprises as
c) mixture of acidic monomers
i) at least one acidic monoester of at least one phosphoric acid of the general formulae I, Ia, Ib and/or II, 2-(meth)acryloyloxyethylphenylic acid phosphate and/or 10-(meth)acryloyloxydecylic acid phosphate, and
(ii) 4-methacryloyloxyethyltrimellitic acid (4-MET) and/or its anhydride (4-META).

15. Dental material according to claim 1, which comprises as
b) at least two different hydrophilic alkylene oxide-based di-functional (meth)acrylate-based crosslinkers selected from i) and ii) or i) and iii) or i), ii) and iii), wherein
i) 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, 1,2-ethanediol diacrylate, diethylene glycol diacrylate, triethylene glycol diacrylate, tetraethylene glycol diacrylate, propylene glycol diacrylate, dipropylene glycol diacrylate, tripropylene glycol diacrylate, and/or tetrapropylene glycol diacrylate, and
(ii) polyethylene glycol di(meth)acrylate and/or polypropylene glycol di(meth)acrylate or mixtures thereof with at least five ethylene glycol or propylene glycol groups, and/or
iii) 1,3-butylene glycol dimethacrylate, hexanediol dimethacrylate, octanediol dimethacrylate, decanediol dimethacrylate, dodecanediol dimethacrylate.

16. (canceled)

17. (canceled)

18. Dental material according to claim 1, wherein the
B) filler component comprises at least one a) dental glass or mixture of dental glasses, and b) optionally comprises at least one inorganic fluoride and/or amorphous metal oxide, mixed oxide, crystalline metal oxide, or silicate,
wherein a) the at least one dental glass comprises aluminosilicate glasses or fluoroaluminosilicate glasses, barium aluminosilicate, barium aluminoborosilicate, strontium silicate, strontium borosilicate, lithium silicate and/or lithium aluminosilicate, dental glasses containing ytterbium fluoride, and mixtures of at least two of the aforementioned dental glasses, and b) comprises ytterbium fluoride, amorphous spherical fillers based on oxide or mixed oxide, quartz, feldspar, and mixtures of at least two of the fillers.

19. Dental material according to claim 1, wherein
e) the diketone of the at least one initiator and/or the at least one initiator system comprises 1,7,7-trimethyl-bicyclo-[2.2.1]-hepta-2,3-dione (champherquinone), phenylpropanedione and at least one co-initiator comprising a tert-amine 2-n-butoxyethyl-4-(dimethylamino)benzoate.

20. Dental material according to claim 1, wherein
A) the monomer component is optionally present in admixture with B) the filler component, the dental material comprising
a) 20 to 80% by weight of at least one at least difunctional urethane (meth)acrylate,
b) 8 to 20% by weight of at least one hydrophilic alkylene oxide-based di-functional (meth)acrylate-based crosslinker comprising i) no urethane (meth)acrylate and/or ii) no aromatic residues, or a mixture of at least two of these crosslinkers,
c) i) 4 to 20% by weight of at least one acidic monoester of at least one phosphoric acid of the general formulae I, Ia, Ib and/or II, 2-(meth)acryloyloxyethylphenylic acid phosphate and/or 10-(meth)acryloyloxydecylic acid phosphate, and
ii) 1 to 10% by weight of at least one carboxylic acid functionalized and/or carboxylic acid anhydride functionalized (meth)acrylate monomer, and or a mixture of i) and ii),
(d) 0.3 to 2.5% by weight of at least one diketone selected from 1,7,7-trimethyl-bicyclo-[2.2.1]-hepta-2,3-dione (champherquinone) and phenylpropanedione, and 0.6 to 2.5% by weight of at least one co-initiator selected from tert-amine and 1,2-methylenedioxybenzene, wherein the mass ratio of diketone to co-initiator selected from tert-amine or 1,2-methylenedioxybenzene is from 1:1.5 to 1:3,
e) 0 to 1% by weight of at least one pigment comprising titanium dioxide, and 0 to 2% by weight of dye, UV stabilizer and/or Vis stabilizer, and
f) 0 to 76% by weight, at least one dental glass,
g) 0 to 10% by weight of inorganic fluoride, such as ytterbium fluoride, metal oxide, mixed oxides and/or silicates,
h) 0 to 1% by weight of at least one pigment comprising titanium dioxide, and 0 to 2% by weight of dye, UV and/or Vis stabilizer, and/or mixtures of at least two of the fillers e) to h), the above components a) to h) giving the total composition of 100% by weight of the dental material.

21. Polymerized dental material obtainable by polymerizing a dental material according to claim 1.

22. A method comprising sealing fissures in enamel and/or dentin using a self-adhesive, radiation-curable material, or producing a radiation-cured, self-adhesive bond to a substrate using a self-adhesive, radiation-curable dental material, wherein the self-adhesive, radiation-curable dental material is one claim 1.

23. Dental material according to claim 10, which comprises 1,6-Bis(methacryloxy-2-ethoxycarbonylamino)-2,4,4-trimethylhexane (UDMA).

24. Dental material according to claim 13, which comprises at least one hydrophilic alkylene oxide-based di-functional (meth)acrylate-based crosslinker, selected from polyethylene glycol di(meth)acrylate and/or polypropylene glycol di(meth)acrylate or mixtures thereof with at least five ethylene glycol or propylene glycol groups.

25. Polymerized dental material according to claim 21, which has a shear bond strength to dentin of greater than or equal to 10 MPa and/or a shear bond strength to enamel of greater than or equal to 12 MPa.

26. Method according to claim 22, which comprises producing a radiation-cured, self-adhesive bond to a substrate using the self-adhesive, radiation-curable dental material, wherein the substrate comprises a metallic substrate, a ceramic substrate, a polymeric molding, and/or a hybrid material.
